(19) United States
(12) Patent Application Publication
(10) Pub. No.: US 2005/0004348 A1
(43) Pub. Date: Jan. 6, 2005

(54) Novel type II Na/Pi cotransporters and type II Na/Pi cotransporter expression regulatory factors

(76) Inventors: Ken-ichi Miyamoto, Tokushima (JP); Hiroko Segawa, Tokushima (JP)

Correspondence Address: FISH & RICHARDSON PC, 225 FRANKLIN ST, BOSTON, MA, 02110, US

(21) Appl. No.: 10/747,032

(22) Filed: Dec. 23, 2003

Related U.S. Application Data
(60) Provisional application No. 60/436,386, filed on Dec. 23, 2002.

(51) Int. Cl. C07K 14/705; C07H 21/04

(52) U.S. Cl. 530/350; 435/69.1; 435/320.1; 435/325; 536/23.5

(57) ABSTRACT

The present invention provides novel type IIc Na/Pi cotransporters. These cotransporters are important Pi transporters that are highly expressed during the growth period from the weaning stage to the adult stage. Furthermore, the present invention provides FGF23 and mutants thereof as factors that regulate the expression of type II Na/Pi cotransporters. FGF23 suppresses Pi reabsorption through suppression of type II Na/Pi cotransporter expression in kidneys. Therefore, FGF23 can be used as a target substance for regulating Pi reabsorption in kidneys. The present invention provides important factors for the development of preventive and therapeutic agents for hyperphosphatemia or hypophosphatemia.

### FIELD OF THE INVENTION

The present invention relates to type II Na/Pi cotransporters, more specifically to novel type II Na/Pi cotransporters expressed in the kidneys of weaning mammals, and expression regulatory factors thereof.

### BACKGROUND OF THE INVENTION

Inorganic phosphate (Pᵢ) is of critical importance to bodily functions, particularly during periods of growth. Kidneys contribute to the maintenance of the positive Pᵢ balance required for growth by reabsorbing a high fraction of the filtered Pᵢ (Pediatr. Nephrol. 16:763-771). The capacity for Na⁺-dependent phosphate cotransport across the luminal brush border membrane of renal proximal tubular cells is greater in juveniles than in adults (Pflugers Arch. Eur. J. Physiol. 394:217-221; and Am. J. Physiol. 257:F268-F274).

Several mammalian renal Na⁺-dependent Pᵢ cotransporters have recently been isolated and characterized (Physiol. Rev. 80:1373-1409) In the kidney cortex, the cDNAs of these transporters can be divided into three types (types I to III) (Physiol. Rev. 80:1373-1409). Type II Na/Pᵢ cotransporters belong to a unique class of Na⁺-coupled cotransport proteins. They can be further subdivided into two subgroups: type IIa and type IIb (Physiol. Rev. 80:1373-1409). Type IIa cotransporters are expressed in the proximal tubule of the kidney, whereas type IIb are expressed in several tissues such as the lungs and small intestine. The functional characteristics of type IIa Na/Pi cotransporters, and the proximal tubular localization and expression of their mRNAs, suggest that these proteins represent a highly likely channel for the proximal tubular Na⁺-dependent entry of Pᵢ (Physiol. Rev. 80:1373-1409).

Age dependence was observed at the level of type IIa Na/Pᵢ cotransporter protein expression (Kidney Int. 50:855-863; and Kidney Int. 49:1023-1026). In addition, a specific type IIa-related Na/Pᵢ cotransporter protein was postulated to account for high Pᵢ transport rates in weaning animals (Am. J. Physiol 273: R928-R933). Evidence for this was obtained by antisense experiments and transport expression in Xenopus oocytes (Pediatr. Nephrol. 16:763-771; and Am. J. Physiol. 273:R928-R933). When mRNA isolatedfrom the kidney cortex of rapidly growing rats was treated with type IIa transporter antisense oligonucleotides, or was depleted of type IIa-specific mRNA using a subtractive hybridization procedure, Na⁺-dependent Pᵢ uptake was still detected in injected oocytes (Nephrol. 16:763-771; and Am. J. Physiol. 273:R928-R933) . The type IIa transporter-depleted mRNA contained an mRNA species that showed partial sequence homology to the type IIa transporter which encodes the message. This conclusion is compatible with the observation that young type IIa (Npt2) knock-out mice lacking the type IIa mRNAand protein still retain their capacity to reabsorb Pᵢ at a rate that cannot be explained by the presence of type I and III Na/Pᵢ transporter (Proc. Natl. Acad. Sci. U.S.A. 95:5372-5377). Accordingly, this strongly suggests the possible existence of transporters as yet unidentified.

Furthermore, the reabsorption of phosphate in kidneys has been studied using transporters that participate in the above-mentioned Pi-uptake, as well as regulatory factors in Pi-reabsorption. For example, the phosphorus (Pi) content of one's diet is amajor regulator of proximal tubular Pi reabsorption, and has been extensively studied using isolated brush-border membrane (BBM) and cell cultures (Murer H., Hernando N., Forster I., and Biber J., “Proximal tubularphosphate reabsorption: molecular mechanisms.” Physiol. Rev. 80: 1373-1409, 2000). These studies demonstrate that changes in proximal Pi reabsorption capacity, as provoked by changes in dietary Pi content, are reflected in altered rates of apical Na+-dependent Pi cotransporters, but not in changes of the apparent Km value for Pi. The regulatory factors for these adaptive systems (dietary Pi) include parathyroid hormone (PTH), vitamin D, growth hormone, thyroid hormone, and calcitonin, but have been suggested to further include agents as yet unidentified (Murer H. et al., 2000, supra).

In autosomal dominant hypophosphatemic rickets (ADHR), a phosphate-wasting disorder, the gene mutated in patients suffering form this disease has been identified as fibroblast growth factor 23 (FGF23), which is a protein that shares sequence homology with fibroblast growth factor 2 (FGF2) (Kruse K., Woelfel D., and Storm T. M., “Loss of renal phosphate wasting in a child with autosomal dominant hypophosphatemic rickets caused by a FGF23 mutation.”, Horm Res 55: 305-308, 2001; and The ADHR Consortium. Autosomal dominant hypophosphatemic rickets is associated with mutations in FGF23. Nat Genet. 26: 345-348, 2000). The FGF23 protein is a novel, secreted protein that consists of 251 amino acids, comprising a putative N-terminal signal peptide (residues 1-24) (Nat. Genet. 26: 345-348, 2000). The missense mutations at 176Arg and 179Arg are responsible for ADHR (Kruse K. et al., 2001, supra; and Nat. Genet. 26: 345-348, 2000, supra) . These amino acid residues are in the consensus proteolytic cleavage sequences represented by “RXXR”. It is possible that mutations at 176Arg and 179Arg prevent proteolytic cleavage, so a large amount of the mutant protein consequently may be secreted as an intact form into a blood circulation (Yamashita T., Konishi M., Miyake A., Inui K., and Itho N., “Fibroblast growth factor (FGF)-23 inhibits renal phosphate reabsorption by activation of the mitogen-activated protein kinase pathway.”, J. Biol. Chem., In press, 2002). Patients with ADHR display many of the clinical and laboratory characteristics observed in patients with oncogenic hypophosphatemic osteomalacia (OHO) (White K. E., Jonsson K. B., Carn G., Hampson G., Spector T. D., Mannstadt M. , Lorenz-Depiereux B., Miyauchi A., Yang I. M., Ljunggren O., Meitinger T., Strom T. M., Juppner H., and Econs M. J., “The autosomal dominant hypophosphatemic rickets (ADHR) gene is a secreted polypeptide overexpressed by tumors that cause phosphate wasting.” J. Clin. Endocrinol. Metab. 86: 497-500, 2001).

White et al. and Shimada et al. demonstrated that FGF23 is a secreted polypeptide overexpressed by tumors that cause Pi wasting (Non-Patent Document 9, White K. E. et al., 2001, supra). Patients with OHO share biochemical and clinical similarities with ADHR patients including hypophosphatemia, decreased or inappropriately normal serum 1, 25(OH)2D3 concentrations, osteomalacia, and reduced tubular maximum reabsorption of Pi (TMP)/glomerular filtration rate (GFR) (KruseK. etal., 2001, supra). Recently, thepresent inventors reported that the administration of naked DNA (FGF23 R176Q) to mice and rats caused the suppression of renal Pi transport activity and phosphaturic effects (WO02052009) . However, the mechanisms by which FGF23 suppresses renal Pi transport activity, for example, what sort of target FGF23 acts on to induce suppression of renal Pi transport, have not been demonstrated.

### SUMMARY OF THE INVENTION

An objective of the present invention is to provide novel type II Na/Pi cotransporter proteins, and to provide factors that regulate the expression of type II Na/Pi cotransporter proteins including the novel transporters.

Upon extensive analysis to accomplish the above-mentioned objective, the present inventors succeeded in isolating a novel type II Na/Pi cotransporter expressed in kidneys, which is different from type IIa Na/Pi cotransporters. Furthermore, the present inventors elucidated that FGF23 suppresses the expression of the type II Na/Pi cotransporter to suppress the reabsorption of Pi in kidneys.

This cotransporter is an important Pi transporter that is highly expressed during the growth period from the weaning stage to the adult stage. Therefore, type IIc Na/Pi cotransporters and genes thereof will be useful in studying, diagnosing, and treating diseases related to defects in Pi reabsorption. Furthermore, type IIc Na/Pi cotransporters and genes thereof are useful in screening for therapeutic agents for diseases relating to defects in Pi reabsorption.

In addition, FGF23, which is a factor that regulates the expression of type II Na/Pi cotransporters, is itself the substance that regulates, or more specifically, suppresses the reabsorption of Pi in kidneys. It can be used as a target substance for regulation of Pi reabsorption in kidneys. Therefore, the present invention provides important factors in the development of preventive and therapeutic agents for hyperphosphatemia or hypophosphatemia.

According to such findings, the present invention provides novel type II Na/Pi cotransporters specifically described below, and expression regulatory factors of type II Na/Pi cotransporters including these novel transporters.

One aspect of the present invention relates to novel type II Na/Pi cotransporters, and this is specifically described below.

[1] A protein selected from any one of the following (a) to (c)
(a) a protein comprising the amino acid sequence of SEQ ID NO: 2;
(b) a protein comprising the amino acid sequence of SEQ ID NO: 4;
(c) a protein comprising the amino acid sequence of SEQ ID NO: 2 or 4, wherein one or more amino acids have been deleted, substituted, or added, wherein the protein comprises Na/Pi cotransporter activity.

[2] A DNA encoding the protein of [1].

[3] The DNA of [2] selected from any one of the following (a) to (c):
(a) a DNA comprising the nucleotide sequence of SEQ ID NO: 1;
(b) a DNA comprising the nucleotide sequence of SEQ ID NO: 3;
(c) a DNA that hybridizes under stringent conditions with a DNA comprising a nucleotide sequence complementary to the nucleotide sequence of SEQ ID NO: 1 or 3.

[4] An oligonucleotide comprising a nucleotide sequence comprising at least 15 continuous nucleotides of the nucleotide sequence of SEQ ID NO: 1 or 3.

[5] A recombinant vector comprising the DNA of [2] or [3].

[6] A transformant obtainable by transforming a host with the vector of [4].

[7] A method of producing an Na/Pᵢ cotransporter, wherein the method comprises culturing the transformant of [5], and collecting a protein comprising Na/Pi cotransport activity from the culture.

[8] An antibody that reacts with the protein of [1].

[9] A method of screening for substances comprising reactivity towards an Na/Pi cotransporter, wherein the method comprises the steps of (A) and (B):
(A) mixing a test substance with the protein of [1]; and
(B) detecting binding between the protein and the test substance.

[10] A method of screening for substances that regulate Na/Pi cotransporter expression, wherein the method comprises the steps of (A) and (B):
(A) adding a test substance to cells expressing the protein of [1], and culturing the cells; and
(B) measuring the protein of [1] expressed in the cells, or an mRNA encoding the protein.

[11] A pharmaceutical composition for treating hypophosphatemia, wherein the composition comprises the DNA of [2].

[12] A method of treatment of hypophosphatemia, wherein the method comprises the step of administering the DNA of [2] to a mammal.

[13] A type II Na/Pi cotransporter expression regulatory factor selected from the proteins of any one of the following (a) to (d):
(a) a protein comprising the amino acid sequence of SEQ ID NO: 6;
(b) a protein comprising the amino acid sequence of SEQ ID NO: 6, wherein arginine at position 176 is replaced with glutamine;
(c) a protein comprising the amino acid sequence of SEQ ID NO: 6, wherein arginine at position 179 is replaced with glutamine; and
(d) a protein comprising an amino acid sequence of any one of the above-mentioned (a) to (c) , wherein one or more amino acids have been deleted, substituted, added, or inserted.

[14] A type II Na/Pi cotransporter expression modulator comprising as an active ingredient a DNA encoding a protein that can regulate expression of a type II Na/Pi cotransporter, wherein the DNA is selected from the following (a) or (b):
(a) a DNA comprising the nucleotide sequence of SEQ ID NO: 5;
(b) a DNA hybridizing under stringent conditions with a DNA comprising a nucleotide sequence complementary to the nucleotide sequence of SEQ ID NO: 5.

[15] The type II Na/Pi cotransporter expression modulator of [14], wherein the DNA is carried in a vector.

[16] A pharmaceutical agent for treatment of hyperphosphatemia, wherein the agent comprises as an active ingredient, the type II Na/Pi cotransporter expression regulatory factor of [13].

[17] A pharmaceutical agent for treatment of hyperphosphatemia, wherein the agent comprises as an active ingredient, the type II Na/Pi cotransporter expression modulator of [14] or [15].

[18] A method of treatment for hyperphosphatemia, wherein the method comprises the step of administering the pharmaceutical agent of [16] or [17] to a patient.

[19] A method of screening for substances that interact with a type II Na/Pi cotransporter expression regulatory factor comprising the following (A) and (B):
(A) reacting a test substance with the type II Na/Pi cotransporter expression regulatory factor of [13];
(B) analyzing the presence or absence of interaction between the test substance and the type II Na/Pi cotransporter expression regulatory factor.

### BRIEF DESCRIPTION OF THE DRAWINGS

FIG. 1. Cloning of Na/Pᵢ cotransporter (type IIc). (a), sequence alignment of type II Na/Pᵢ cotransporters. The deduced amino acid sequence of type IIc Na/Pᵢ cotransporter (human) is shown aligned with those of types IIa, IIb, and IIc cotransporters. Residues identical in at least two sequences are shaded. Lines under the sequences show predicted transmembrane regions of type IIc Na/Pᵢ cotransporter, numbered 1 to 8. In type IIc Na/Pᵢ cotransporter, putative N-linked glycosylation sites are marked by the # sign. Putative protein kinase C-dependent phosphorylation sites are located at residues 24, 152, 481, and 581 (labeled with *) The residue numbers are indicated beside the aligned sequences. (b), Northern blotting analysis in human tissues. High stringency Northern hybridization analysis using a human type IIc probe was performed against poly(A)⁺ RNA from human tissues. (c), Northern blotting analysis in rat tissues. High stringency Northern hybridization analysis using a rat type IIc probe was performed against poly(A)⁺ RNA from rat tissues. (d), developmental changes in rat renal type IIc mRNA levels. Lane 1, 5 day-old; lane 2, 15 day-old; lane 3, 22 day-old; and lane 4, 60 day-old.

FIG. 2. Characterization of the type IIc Na/Pᵢ cotransporter in Xenopus oocytes. (a), oocytes injected with either water (open bar), cRNA of human type IIc Na/Pᵢ cotransporter (closed bar), or cRNA of rat type IIa Na/Pᵢ cotransporter (open bar) (10) were assayed after 2 days for uptake of Pᵢ (100 μM) in 96 mM NaCl medium (n=8 experiments) Values are means ±S.E. (b) , ion dependence of Pᵢ transport in oocytes expressing human type.IIc Na/Pᵢ cotransporter. The uptake of 100 μM Pᵢ measured in the standard uptake solution (Na) was not increased in the Na⁺-free uptake solution in which Na⁺ was replaced with choline. In contrast, it was not altered in the Cl⁻-free uptake solution in which Cl⁻ was replaced with gluconate. Values are means ±S.E. (n=3). (c) , Pᵢ concentration dependence of human type IIc Na/Pᵢ cotransporter-mediated Pᵢ uptake. The type IIc Na/Pᵢ cotransporter-mediated Pᵢ uptake was measured at 3, 10, 30, 100, 300, and 1000 μM Pᵢ in standard uptake solution and plotted against the Pᵢ concentration. The Pᵢ uptake was saturable and fit the Michaelis-Menten curve. Values are means ±S.E. (n=6 experiments). (d), sodium concentration dependence of type IIc Na/Pᵢ cotransporter-mediated Pᵢ uptake. The type IIc Na/Pᵢ cotransporter-mediated Pᵢ uptake was measured at 10, 25, 50, 75, and 100 mM sodium. Choline was used for isosmotic ionic replacement. Values are means ±S.E. (n=5 experiments). (e), pH dependence of type IIc Na/Pᵢ cotransporter-mediated Pᵢ uptake. The type IIc cotransporter-mediated uptake of Pᵢ (100 μM) was measured in the standard uptake solution at various pH values. The uptake value was greatest at pH 7.5. Values are means ±S.E. (n=5 experiments). (a) Pᵢ uptake (pmol/oocyte/min), and water, type IIc and type IIa; (b) Pᵢ uptake (pmol/oocyte/min), and Na, choline and gluconate; (c) Pᵢ uptake (pmol/oocyte/min) ; (d) Pᵢ uptake (pmol/oocyte/min) , and Na⁺ (mM) ; (e) Pᵢ uptake (pmol/oocyte/min) , and pH 5.5, pH 6.5 and pH 8.5.

FIG. 3. Voltage-dependent Pᵢ-induced currents in an oocytes expressing type IIa and type IIc Na/Pᵢ cotransporters. (A), time course changes in membrane current during stimulation by Pᵢ. Pᵢ perfusion was performed with the indicated concentration and for the indicated times. The holding membrane potential was −60 mV. (B), current-voltage curves. The current-voltage relationship was recorded by the voltage clamp protocol before stimulation, during perfusion with Pᵢ, and after washout of Pᵢ. C, Pᵢ dose-response relation for membrane currents. The membrane current was measured at Vₘ=−60 mV. Values were recorded at the steady-state response of membrane current and are means ±S.E. (n=6). (I), oocytes were injected with cRNA of type IIa Na/Pᵢ cotransporter. (II), oocytes were injected with cRNA of type IIc Na/Pᵢ cotransporter. (A) Current (nA), Pi concentration, and time (min). (B) Before Pi, after washout, 1 mM Pi, membrane potential (mV), before Pi. (C) Current change (nA) and Pi concentration (μM).

FIG. 4. Western blotting analysis under reducing conditions. (a) , Western blotting analyses were performed on BBMVs prepared from rat kidney in the presence of 2-mercaptoethanol. Lane 1, type IIc antibodies; lane 2, results from peptide absorption experiments. (b) The type IIc antibodies did not react with the type IIa Na/Pᵢ cotransporter. To generate FLAG-tagged type IIc transporter cDNA, PCR amplification was performed with the rat type IIc clone as a template. Fragments were subcloned into the pFLAG-CMV-2 expression vector (Sigma) for transient transfection. Total protein homogenates from COS-7 cells are shown. Lane 1, control cells (transfected with empty vector); lane 2, transient transfected with FLAG-type IIc transporter vector. The type IIa (lane 3) or IIb (lane 4) Na/Pᵢ cotransporter cDNA was subcloned for the pCDNA3.1(+) for transient transfection. The membranes were treated with diluted affinity-purified anti-type IIc (1:1,000) Na/Pᵢ cotransporter antibody. Findings indicate that the type IIc antibodies are not reacted with type IIa and IIb Na/Pᵢ cotransporters. (c), developmental changes in rat renal type IIc protein levels. Renal BBMVs from each aged rat were prepared, and 20 μg of the protein was analyzed by Western blot analysis. Lane 1, 5 day-old; lane 2, 15 day-old; lane 3, 22 day-old; and lane 4, 60 day-old. (d), relative intensity of the type IIc transporter protein in developmental rats. **, p<0.01. (e) , effects of dietary Pᵢ on the amounts of type IIc protein. Brush-border membrane vesicles were isolated from 40-day-old rats fed the test diet for 6 days. Lane 1, low Pᵢ (0.02%) diet; lane 2, control Pᵢ (0.6%) diet; lane 3, high Pᵢ (1.2%) diet. **, p<0.01. (f), relative intensity of the type IIc transporter protein in rats fed a low, normal, or high Pᵢ diet.

FIG. 5. Localization of type IIc-immunoreactive protein in weaning and adult kidneys. Type II Na/Pᵢ cotransporter proteins detected by diaminobenzidine staining using rabbit anti-type IIc antibodies ((a) and (b)) or rabbit anti-type IIa antibodies ((c) and (d)) in cryostat sections of weaning rat kidneys. The type IIc transporter protein in adult kidneys is shown in panels (e) and (f) At higher magnification, type IIc (g) and type IIa (h) antibody-mediated immunoreactivities are shown.

FIG. 6. Hybrid depletion of type II Na/Pᵢ cotransporter in Xenopus oocytes. Pᵢ uptake in oocytes injected with renal poly(A)⁺ RNA from weaning and adult rat kidneys. Hybrid depletion analyses using antisense oligonucleotide were performed as described under “Experimental Procedures”. (a) and (c), poly(A)⁺ RNA from adult rat kidney (60 day-old). (b) and (d), poly(A)⁺ RNA from weaning rat kidney (22 day-old) . Values are mean ±S.E. (n=8 to 10 oocytes) . **, p<0.01; *, p<0.05. (a) pmol/oocyte/minute; and adult rat kidney poly(A)⁺ RNA, antisense type IIa, sense type IIa, and control. (b) weaning rat kidney poly(A)⁺ RNA, antisense type IIa, sense type IIa, and control. (c) pmol/oocyte/minute; and adult rat kidney poly(A)⁺ RNA, antisense type IIc, sense type IIc, and control. (d) weaning rat kidney poly(A)⁺ RNA, antisense type IIc, sense type IIc, and control.

FIG. 7 shows the expression levels of FGF23 and such in livers derived from Pi-depleted rats transfected with human FGF23 naked DNA and such. n=4, values are means ±S.E., and p<0.01**.

FIG. 8 shows the results of measuring sodium-dependent Pi transporter activity in renal BBMVs isolated from rats given a diet low in Pi, based on the uptake of radiolabeled Pi. (a) shows the influence of the low Pi diet on Na/Pi cotransporter activity in rat kidneys. The data is shown as nmol/mg protein/minute. (b) shows the result of Western blotting analysis of type I, type IIa, and type IIc Na/Pi cotransporters. LP: low Pi diet, CP: control Pi diet.

FIG. 9 shows the result of measuring sodium-dependent Pi transporter activity in renal BBMVs of rats transfected with pCGF23 (wild-type FGF23), pCGFM2 (FGF23 R176Q mutant), or an empty plasmid (Mock) , based on the uptake of radiolabeled Pi. The data are means ±S.E., where n=4, and p<0.05*.

(a) Pi transport activity in 1 minute in BBMVs, and (b) Pi transport activity in BBMVs over a time course.

FIG. 10 shows the results of measuring the expression levels of type I, type IIa, and type IIc Na/Pi cotransporters in FGF23 (R176Q)-treated Pi-depleted rats by Western blotting analysis. The upper panels show the results of Western blot analysis, and the lower panels show the relative strength of type I, type IIa, and type IIc transporter proteins in BBMVs. (a) type I, (b) type IIa, and (c) type IIc Na/Pi cotransporters. Mock: mock-plasmid injected control. Two independent experiments (n=4) were performed and the data are indicated in the respective panels. The data are shown as means ±S.E., where n=4, p<0.01**, and p<0.05*.

FIG. 11 shows the results of analyzing the transcription level of type IIa and type IIc transporter genes in kidney cortexes by Northern blotting. Three micrograms of poly(A)+RNA was loaded onto each lane (left pannels) . Relative intensities of type IIa and type IIc transcripts are shown in the right panels. Data are means ±S.E. (n=4). GAPDH was used as an internal control.

FIG. 12 shows the results of immunohistochemical analysis of type IIa Na/Pi cotransporter in renal proximal tubular cells of rat kidneys. Mock-plasmid injected control ((a) and (b)), and FGF23 (R175Q) DNA ((c) and (d)). Magnification: 40 times ((a) and (c)) 100 times ((b) and (d)).

FIG. 13 shows the results of immunohistochemical analysis of type IIc Na/Pi cotransporter in rat kidneys. Mock-plasmid injected control ((e) and (f)). FGF23 (R176Q) DNA ((g) and (h)). Magnification: 40 times ((e) and (g)), and 100 times ((f) and (h)).

FIG. 14 shows the results of investigating the influence of FGF23Rl79Q on type IIa NaPi cotransporter expression. (A): Results of a Western blot analysis of type IIa NaPi cotransporter expression in the renal proximal tubular brush border membrane vesicles of the kidneys of the FGF23R179Q expression plasmid-administered group and the mock-plasmid injected control (MOCK) group are shown. (B): Quantitative graph of the Western blot analysis of (A) is shown. MOCK: renal brush border membrane vesicles of the MOCK plasmid-administered group, FGF23: renal brush border membrane vesicles of the FGF23 mutant (FGF23R179Q) expression plasmid-administered group.

FIG. 15 shows the results of investigating the influence of FGF23R179Q on type IIc NaPi cotransporter expression. (A): Results of a Western blot analysis of type IIc NaPi cotransporter expression in renal proximal tubular brush border membrane vesicles of the kidneys of the MOCK-administered group and the FGF23R179Q-administered group are shown. (B): Quantitative graph of the Western blot analysis of (A) is shown. MOCK: renal brush border membrane vesicles of the MOCK plasmid-administered group, FGF23: renal brush border membrane vesicles of the FGF23 mutant (FGF23R179Q) expression plasmid-administered group.

FIG. 16 shows the results of analyzing the influence of FGF23R179Q on type I NaPi cotransporter expression. (A): Results of a Western blot analysis of type I NaPi cotransporter expression in the renal proximal tubular brush border membrane vesicles of the kidneys of the MOCK-administered group and the FGF23R179Q-administered group are shown. (B) : Quantitative graph of the type I NaPi cotransporter of the Western blot analysis of (A) is shown. MOCK: renal brush border membrane vesicles of the MOCK plasmid-administered group, FGF23: renal brush border membrane vesicles of the FGF23 mutant (FGF23R179Q) expression plasmid-administered group.

FIG. 17 shows phosphorus transfer activity due to FGF23R179Q administration when using (A) kidney and (B) small intestinal brush border membrane vesicles. MOCK: kidney and small intestinal brush border membrane vesicles of the MOCK plasmid-administered group, FGF23: kidney and small intestinal brush border membrane vesicles of the FGF23 mutant (FGF23R179Q) expression plasmid-administered group.

FIG. 18 shows the results of analyzing the influence of FGF23R179Q on type IIa NaPi gene expression in kidneys. (A) : Results of a Northern blot analysis of type IIa NaPi cotransporter mRNA are shown, and (B): quantitative graph of type IIa NaPi cotransporter mRNA Northern blot analysis of (A) is shown. MOCK: renal total RNA of the MOCK plasmid-administered group, FGF23: renal total RNA of the FGF23 mutant (FGF23R179Q) expression plasmid-administered group.

FIG. 19 shows the results of analyzing the influence of FGF23R179Q on type IIa NaPi cotransporter expression. (A): Results of immunohistochemical staining analysis of kidneys of the MOCK-administered group are shown. It indicates clear staining of renal brush border membranes. (B): Results of immunohistochemical staining analysis of kidneys of the FGF23R179Q-administered group are shown. It indicates that renal brush border membranes were stained overall, but it is apparent that staining is weak compared to those of (A).

FIG. 20 shows the results of analyzing the influence of FGF23R179Q on type IIc NaPi cotransporter expression. (A): Results of immunohistochemical staining analysis on kidneys of the MOCK-administered group are shown. It shows overall staining of renal brush border membranes. (B): Results of immunohistochemical staining analysis of kidneys of the FGF23R179Q-administered group are shown. It shows that staining is weak overall compared to those of (A).

### DETAILED DESCRIPTION OF THE INVENTION

The present invention provides novel type II Na/Pi cotransporters (hereinafter, referred to as “type IIc Na/Pi cotransporters”) . The Na/Pi cotransporters of the present invention have similarity to the conventional type IIa sequences from rats and humans, but were obtained as substances having different biological and chemical characteristics. The type II Na/Pi cotransporters of the present invention share common aspects with the conventional type IIa in that they similarly reabsorb Pi in a sodium ion-dependent manner in the kidney, but differ in that they are electroneutral. They also show differences in the timing of expression, so that they are expressed most strongly during the weaning stage and also expressed in adults. Specifically, the cotransporters of the present invention differ from the conventional type IIa in that they are important cotransporters during the growth period, and their expression is increased from the weaning stage. Such cotransporters are useful as research reagents for elucidating the Pi reabsorption mechanism during the growth period, and will be useful in the medical field including genetic diagnosis and gene therapy of patients having defects in Pi reabsorption function.

Such type IIc Na/Pi cotransporters of this invention include a protein comprising the amino acid sequence shown in SEQ ID NOs: 2 or 4. However, existence of mutants having the same function is predicted for the protein, and mutants having the same function can be produced by artificially and appropriately modifying the amino acid sequence of the protein. Therefore, as long as Na/Pi cotransport activity is present, the cotransporters of this invention include proteins comprising an amino acid sequence, in which one or more of the amino acids of SEQ ID NO: 2 or 4 are deleted, substituted or added.

Modification of the amino acid sequence of a protein can be performed by modifying a nucleotide sequence of a DNA encoding a protein by well known means such as site specific mutagenesis, and expressing the DNA whose nucleotide sequence has been modified. Furthermore, the number of mutated amino acids and mutation sites in the protein are not limited as long as its function is retained. For example, amino acids belonging to each of the following groups have properties similar to each other within the group. Even if these amino acids are substituted with other amino acids within the group, the essential functions of the protein are usually not lost. Such amino acid substitution is called conservative substitution, and is well known as a technique for modifying the amino acid sequence while retaining the function of the protein.

Non-polar amino acids: Ala, Val, Leu, Ile, Pro, Met, Phe, and Trp

Uncharged amino acids: Gly, Ser, Thr, Cys, Tyr, Asn, and Gln

Acidic amino acids: Asp and Glu

Basic amino acids: Lys, Arg, and His

Whether these modified proteins and analogous sequences obtained from nature have Na/Pi cotransport activity or not can be confirmed by following the EXAMPLES in the present description.

The present invention provides DNAs encoding the above-mentioned type IIc Na/Pi cotransporters. Examples of the DNAs of the present invention include a DNA comprising the nucleotide sequence of SEQ ID NO: 1 or 3. Also in terms of genes, existence of genes comprising different nucleotide sequences while encoding the same amino acid sequence or mutants retaining the same function is predicted. In addition, genes encoding the same product or mutants having the same function can be produced by artificially modifying the nucleotide sequence. Therefore, the DNAs of the present invention include DNAs analogous to the nucleotide sequence of SEQ ID NO: 1 or 3 as long as they encode a protein that retains Na/Pi cotransport function. Herein, DNAs having an analogous nucleotide sequence include DNAs that hybridize under stringent conditions with a DNA comprising a nucleotide sequence that is complementary to the nucleotide sequence of SEQ ID NO: 1 or 3; or DNAs comprising a nucleotide sequence having 90% or more, preferably 95% or more, more preferably 98% or more, and even more preferably 99% or more homology to the nucleotide sequence of SEQ ID NO: 1 or SEQ ID NO: 3.

Stringent conditions refer to conditions for washing, which are ordinarily “1×SSC, 0.1% SDS, and 37° C.” or so, more stringently “0.5×SSC, 0.1% SDS, 42° C.” or so, and even more stringently “0.1×SSC, 0.1% SDS, 55° C.” or so. Furthermore, homology can be calculated by methods such as the ClustalW method.

The DNAs that encode type IIc Na/Pi cotransporters can be obtained by conventional methods based on the nucleotide sequences disclosed in the present specification. For example, by screening a cDNA library derived from renal tubular cells of a weaning mammal using DNAs comprising the nucleotide sequence of SEQ ID NO: 1 or 3 as a probe, cDNAs encoding a type IIc Na/Pi cotransporter can be isolated. Furthermore, by performing PCR using this cDNA library as a template, and using primers constructed based on the nucleotide sequence of SEQ ID NO: 1 or 3, DNAs encoding a type IIc Na/Pi cotransporter can be amplified. The amplification products are cloned based on conventional methods.

Furthermore, the present invention also provides vectors comprising a DNA encoding a type IIc Na/Pi cotransporter. Insertion into vectors is beneficial for amplification of a DNA encoding a type IIc Na/Pi cotransporter, transfection of the DNA into a host, expression of a type IIc Na/Pi cotransporter, and such. There are no particular limitations on the vectors that may be used in the present invention, and may be selected appropriately according to the objective (amplification, expression, etc.) and the transformed host. Specifically, examples include vectors derived from mammals (for example, pcDNA3 (Invitrogen), andpEGF-BOS (Nucleic Acids. Res., 18(17), p.5322, 1990), pEF, pCDM8, pCXN, vectors derived from insect cells (for example, “Bac-to-BAC baculovirus expression system” (Invitrogen) , and pBacPAK8) , expression vectors derived from plants (for example, pMH1 and pMH2), vectors derived from animal viruses (for example, pHSV, pMV, pAdexLcw) , vectors derived from retroviruses (for example, pZIPneo) , vectors derived from yeast (for example, “Pichia Expression Kit” (Invitrogen), pNV11, and SP-Q01), vectors derived from Bacillus subtilis (for example, pPL608, and pKTH50), and Escherichia coil vector (M13 vector, pUC vector, pBR322, pBluescript, and pCR-Script). Among them, for purposes of gene therapy, preferably vectors expressible in mammalian cells, more preferably expression vectors are used. For such vectors that may be used for purposes of gene therapy, the above-mentioned vectors derived from mammalian animals, vectors derived from animal viruses, vectors derived from retroviruses, and such are effective.

Genetic engineering methods such as insertion of a DNA into a vector can be performed according to the methods described in the literature (Maniatis et al., Molecular Cloning: A Laboratory Manual, Cold Spring Harbor Laboratory, 1989).

The DNAs encoding a type IIc Na/Pi cotransporter of this invention are not only useful in their full-length form, but portions thereof can also be used as primers or probes, as described above, for expression analysis of type IIc Na/Pi cotransporter genes, cloning, genetic diagnosis, and such. In the present invention, the term “portion” means an oligonucleotide comprising a length that may function as primers or probes. The length of a nucleotide sequence that may function as a primer includes at least 10 mer, preferably at least 15 mer, and more preferably at least about 20 mer to about 30 mer. Furthermore, probes include nucleotide sequences of at least 10 mer, preferably at least 15 mer, and more preferably at least about 20 mer to about 30 mer. The specificity can be increased further when using those with a longer chain or when using the entire DNA.

Furthermore, partial sequences of the DNAs of this invention can be used for antisense method, RNAi, and such. The antisense method means that when an oligonucleotide having a sequence complementary to the target gene exists in a cell, translation and transcription are inhibited by pairing of the antisense (antisense DNA, antisense RNA, etc.) and the target gene (target mRNA, target DNA, etc.) and the expression of the target gene is suppressed. Therefore, expression of a type IIc cotransporter, which is a target DNA in a cell, may be inhibited by using a partial sequence of a DNA of this invention as the antisense oligonucleotide, inserting this antisense oligonucleotide into a vector, and transforming the cell using this vector.

Alternatively, a target gene can be inhibited by using RNA interference (RNAi). RNAi refers to a phenomenon in which when a double stranded RNA (dsRNA) is transferred into a cell, mRNA in the cell corresponding to that RNA sequence is specifically degraded, so it is not expressed as a protein. In the case of RNAi, normally, a double stranded RNA is used, but it is also possible to use a double strand formed within a self-complementary single-stranded RNA. The region forming the double strand may form a double strand in the entire region, or form a single strand in parts of the region (for example, both ends, or one of the ends). Oligo RNA used for RNAi is often an RNA of 10 bp to 100 bp, and is usually an RNA of 19 bp to 23 bp. Therefore, expression of a type IIc cotransporter gene in a cell can be inhibited by inserting a partial sequence of the type IIc cotransporter DNA of this invention, which is constructed to form a double-stranded RNA within a cell, into a DNA construct or a vector of this invention, and transforming the cell with the DNA construct or the vector. The RNAi method can be performed according to Nature, Vol. 391, p.806, 1998; Proc. Natl. Acad. Sci. USA, Vol. 95, p.15502, 1998; Nature, Vol. 395, p.854, 1998; Proc. Natl. Acad. Sci. USA, Vol. 96, p.5049, 1999; Cell, Vol. 95, p.1017, 1998; Proc. Natl. Acad. Sci. USA, Vol. 96, p.1451, 1999; Proc. Natl. Acad. Sci. USA, Vol. 95, p.13959, 1998; Nature Cell Biol., Vol. 2, p.70, 2000; and such.

The present invention also provides DNAs encoding type IIc Na/Pi cotransporters or transformants transformed by vectors comprising the DNAs. These transformants will be useful for producing a type IIc Na/Pi cotransporter of the present invention. There are no particular limitations on the host cells used for transformation, and can be selected according to the objective. For example, hosts for expressing proteins include bacterial cells (for example, Streptococcus, Staphylococcus, E. coli, Streptomyces, and Bacillus subtilis) , fungal cells (for example, yeast and Aspergillus) , insect cells (for example, Drosophila S2 and Spodoptera SF9), animal cells (for example, CHO, COS, HeLa, C127, 3T3, BHK, HEK293, and Bowes melanoma cells) , and plant cells. The method for introducing vectors into cells can be performed by selecting from methods such as calcium phosphatemethod (Virology, Vol. 52, p.456, 1973) , DEAEdextranmethod, a method using cationic liposome DOTAP (Roche Diagnostics), electroporation method (Nucleic Acids Res., Vol. 15, p.1311, 1987), lipofection method (J. Clin. Biochem. Nutr., Vol. 7, p.175, 1989), method of introducing by viral infection (Sci. Am., p.34, 1994) , and particle gun. These specific methods can be performed by following methods such as those described in the literature (Maniatis et al., Molecular Cloning: A Laboratory Manual, Cold Spring Harbor Laboratory, 1989).

The present invention provides methods of producing type IIc Na/Pi cotransporters using the above-mentioned transformants. More specifically, the above-mentioned transformants are cultured, and type II Na/Pi cotransporters expressed by the transformants are collected from the culture. Herein, culturing should be performed under conditions in which the transformants express the proteins of this invention. When the proteins expressed herein are secreted to the outside of the cells, the proteins are collected from the culture supernatants; and when the proteins are produced inside the cells, the proteins are collected from the cell lysates. Protein purification can be performed by appropriately combining conventionally used purification methods (chromatography, electrophoresis, gel filtration, etc.). Furthermore, to facilitate the purification of the proteins of this invention, the proteins of the present invention are expressed, for example, as fusion proteins with GST or His tag. Furthermore, corresponding to the selected tags, each of them can be purified using a glutathione sepharose column or a nickel sepharose column.

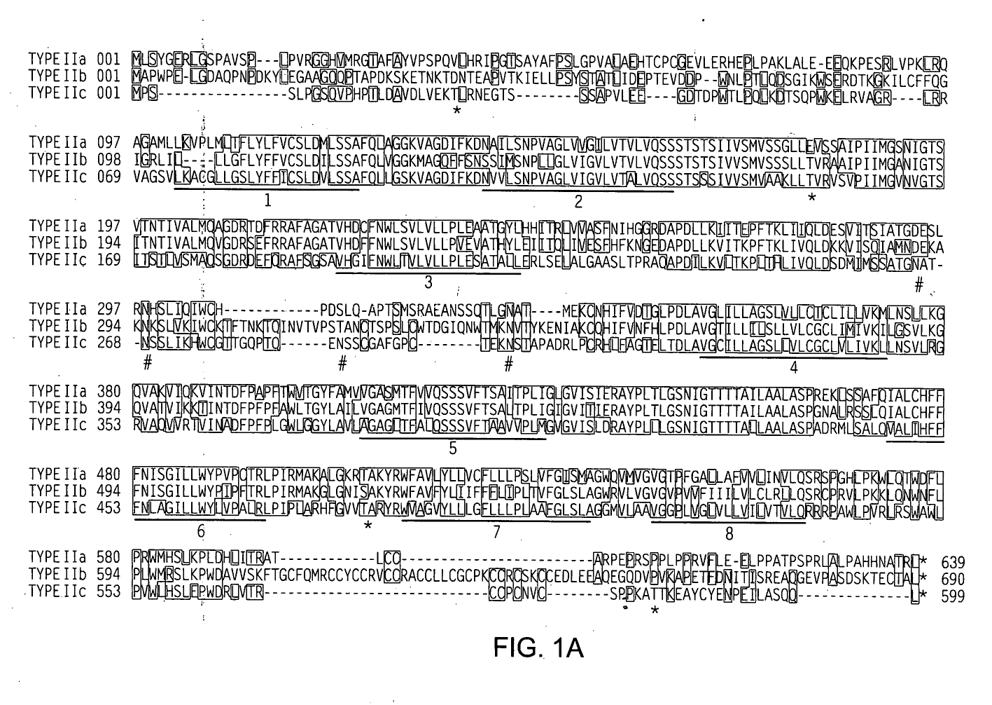

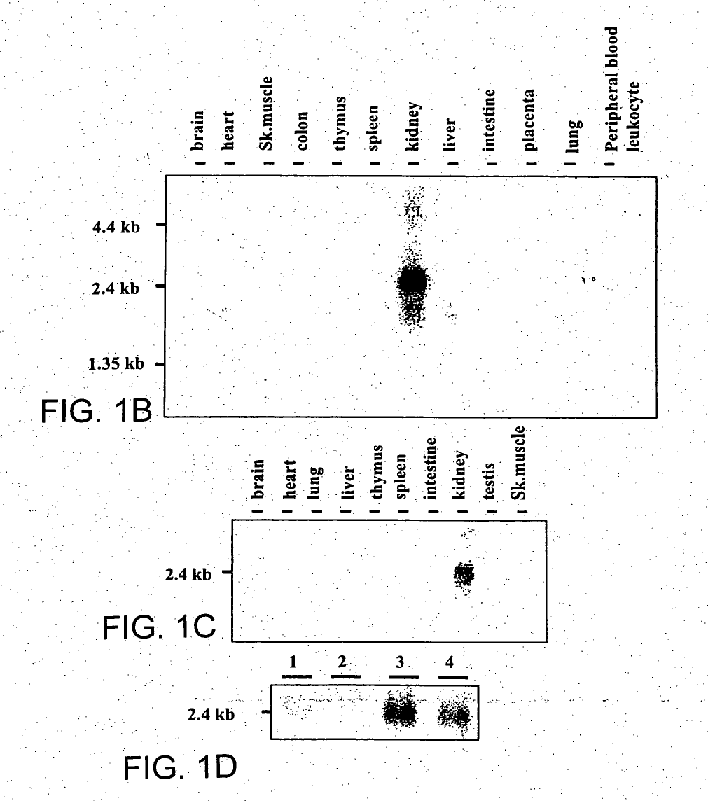

The present invention provides specific antibodies against type IIc Na/Pi cotransporters. These antibodies will be useful as reagents for detecting and diagnosing whether a type IIc Na/Pi cotransporter is expressed in vivo, for example, in kidneys. The type of antibodies may be polyclonal antibodies, monoclonal antibodies, chimeric antibodies, or human antibodies. Antibodies can be produced using the entire type IIc Na/Pi cotransporter protein (for example, the full length protein of SEQ ID NO: 2 or 4), or a partial peptide thereof. For example, as the partial peptide, the use of a region in which the sequence of the type IIc Na/Pi cotransporter is different from the type IIa Na/Pi cotransporter as shown in FIG. 1 will be advantageous when producing type IIc-specific antibodies. When the partial peptide is used as an antigen, it is generally preferable to comprise at least 6 amino acids in order for the peptide to maintain antigenicity.

Antibodies can be produced by conventional methods. For example, regarding polyclonal antibodies, proteins or peptides are innoculated several times with an interval into animals such as mice, rats, rabbits, guinea pigs, and pigs, and the antibodies are prepared from the serum of immunized animals with increased antibody titer. Monoclonal antibodies are prepared by fusing myeloma cells with antibody producing cells obtained from the spleen or lymph nodes of the above-mentioned immunized animals, and selecting hybridomas that produce antibodies showing strong specificity to the proteins of this invention.

Qualitative and quantitative analyses of proteins of the present invention in a biological sample can be performed by immunological methods that use the antibodies obtained by the present invention. Known methods such as immunohistological staining, enzyme immunoassay, aggregation assay, competition assay, and sandwich method can be applied as immunological methods on samples obtained by appropriately treating biological samples as necessary, for example by separation and extraction of cells. Immunohistological staining may be performed by direct methods using labeled antibodies, by indirect methods using labeled antibodies against the antibodies, and such. Any of the conventional labeling substances such as fluorescent substances, radioactive substances, enzymes, metals, and pigments can be used as labeling agents.

The present invention provides methods of screening for substances that influence type IIc Na/Pi cotransporter expression. More specifically, cell lines expressing a type IIc Na/Pi cotransporter, for example, cell strains derived from renal tubular cells in the weaning stage, are selected by northern blotting, RT-PCR, and such. Furthermore, selection can be carried out by the fluorescent antibody method, enzyme antibody method, and such, using antibodies obtained by the method mentioned above. The selected cells are cultured in the presence of a test substance, mRNA expression level is quantified by northern blotting, slot blot hybridization, RT-PCR and such, or the expression level is quantified by fluorescent antibody method, enzyme antibody method, and such, and the influence of the test substance on type IIc Na/Pi cotransporter expression is measured. Furthermore, there are no particular limitations on the test samples. Examples include natural or synthetic compounds, various kinds of organic compounds, natural or synthetic sugars, proteins, peptides, expression products of gene libraries, cell extract, or microbial components.

Furthermore, the following measures can be taken so that large amounts of substances can be screened more easily. Clones that hybridize to the cDNA 5′ region of the proteins of this invention are selected from a human DNA library. This is inserted into an appropriate promoter screening system to select clones having promoter activity. To construct reporter genes, a DNA selected herein, carrying a promoter region of a protein of this invention, is inserted upstream of a DNA encoding an enzyme such as luciferase and alkaline phosphatase, whose activity can be measured easily. Cell strain allowing measurement of the activity of a promoter that expresses the protein of this invention is established by transfecting this reporter gene into cells such as HeLa cells that can be cultured with appropriate resistant genes such as Neo^{r} and hyg^{r}, then selecting with pharmaceutical agents corresponding to the resistant gene. Substances influencing the expression of the proteins of this invention are screened by measuring the activities of the introduced enzymes by acting test substances on this cell strain. Herein, substances that positively regulate the expression of type IIc Na/Pi cotransporters promote reabsorption of Pi through increase in type IIc Na/Pi cotransporter expression, and allow blood phosphorus concentration to be elevated. On the contrary, substances that negatively regulate the expression of type IIc Na/Pi cotransporters suppress reabsorption of Pi through suppression of type IIc Na/Pi cotransporter expression, and allow blood phosphorus concentration to be decreased. Therefore, the former substances may become candidate substances for therapeutic agents for hypophosphatemia, and the latter may become candidate substances for therapeutic agents for hyperphosphatemia. An example of substances having the function of the latter is FGF23, which will be described later. FGF23 may be used as a control for this screening method.

Furthermore, the present invention provides methods of screening for substances that interact with type IIc Na/Pi cotransporters. The present screening methods comprise the steps of mixing a type IIc Na/Pi cotransporter and a test substance, and detecting binding between the protein and the test substance. There are no particular limitations on the form of the protein used in this screening, and it may be a purified or crude protein, a soluble protein, a protein bound to a carrier, a protein bound to a membrane, or such. There are no particular limitations on the test samples, and similarly to the above-mentioned screening, examples include natural or synthetic compounds, various types of organic compounds, natural or synthesized sugars, proteins, peptides, expression products of gene libraries, cell extract, or microbial components. The interaction after mixing the proteins can be detected by conventional methods, such as immunoprecipitation method, pull down assay, two-hybrid method, and BIAcore. Furthermore, whether the substance interacting in this screening method suppresses or enhances type IIc Na/Pi cotransporter activity may be analyzed further based on Na/Pi cotransporter activity. As a result of this analysis, substances that may raise type IIc Na/Pi cotransport activity promote reabsorption of Pi through increased expression of type IIc Na/Pi cotransporter, and the blood phosphorus concentration can be increased. On the other hand, as a result of the interaction, substances that act in an inhibitory manner toward type IIc Na/Pi cotransporter activity suppress reabsorption of Pi, and the blood phosphorus concentration can be decreased. Therefore, the former substances may become candidate substances for therapeutic agents for hypophosphatemia or diseases caused by hypophosphatemia, and the latter may become candidate substances for therapeutic agents for hyperphosphatemia or diseases caused by hyperphosphatemia (secondary hyperparathyroidism, renal osteodystrophy of a patient on long-term dialysis, and such).

The present invention also provides gene therapies using DNAs encoding type IIc Na/Pi cotransporters. Gene therapy has the objective of correcting mutated genes, and is a method of performing therapy of a disease by transferring normal genes into cells of a patient from the outside and altering the phenotype of the cells. Gene therapy is considered to be effective not only for treatment of genetic diseases, but also for other diseases where the causative gene is normal but the disease develops in a secondary manner. Therefore, this is effective not only for treating patients having hypophosphatemia due to a defect in the type IIc Na/Pi cotransporter gene, but also for treating patients who have developed hypophosphatemia due to other factors. In the gene therapy of the present invention, the above-mentioned type IIc Na/Pi cotransporter gene is administered to a patient as it is. Alternatively, it is administrated as the above-mentioned mammalian expression vector appropriately harboring it. Gene therapy is categorized into methods in which a gene is incorporated into a cell by transferring a gene directly into the body (in vivo method); and methods in which cells are taken from a patient, a gene is transfected into the cells outside the body, and then these cells are retransplanted into the patient (ex vivo method). The method of the present invention may adopt either method. Expression of the administered gene in vivo supplements a complete type IIc Na/Pi cotransporter and the action of this exogenous type IIc Na/Pi cotransporter enables increase of blood phosphate concentration.

Diseases and conditions likely to cause lowering of blood Pi concentration include osteomalacia, hyperparathyroidism, abnormal vitamin D metabolism, malabsorption syndrome, fasting, glucosuria, and chronic alcoholism. Furthermore, hypophosphatemia may occur because of complications and side effects due to treatment. Such treatments that may accompany hypophosphatemia include renal transplantation, glucagon administration, and glucose administration. Therefore, in order to treat lowering of blood phosphate concentration due to these diseases and treatments, treatments using the above-mentioned type IIc Na/Pi cotransporter genes will become effective.

(2) Type II Na/Pi Cotransporter Expression Regulatory Factor

The present invention provides novel type II Na/Pi cotransporter regulatory factors. The present inventors have reported that human FGF23 lowers the blood phosphorus concentration, and by further studies, found out that this mechanism involves suppression of type II Na/Pi cotransporter expression by FGF23, followed by suppression of reabsorption of phosphorus in kidneys, and a consequent decrease of blood phosphorus concentration. More specifically, the present invention is based on the finding that human FGF23 and mutants thereof function as type II Na/Pi cotransporter expression regulatory factors, and provide human FGF23 and mutants thereof as type II Na/Pi cotransporter expression regulatory factors.

FGF23, which is a type II Na/Pi cotransporter expression regulatory factor, comprises the amino acid sequence of SEQ ID NO: 6, but this invention is not limited thereto, and can include proteins comprising a similar sequence that may regulate the expression of type II Na/Pi cotransporters. As described above, FGF23 in particular, has a region from the 176th residue to the 179th residue of SEQ ID NO: 6, which is cleaved by proteases. FGF23 mutants having a mutation in this cleavage region so that it is not digested by proteases may maintain type II Na/Pi cotransporter expression suppression activity of FGF23 even in the presence of proteases. Examples of such mutants include a mutant in which 176th arginine residue is substituted with glutamine, a mutant in which 179th arginine residue is substituted with glutamine, and a mutant in which 179th arginine residue is substituted with tryptophan in the amino acid sequence of human FGF23 protein (hereinafter, they are referred to as “R176Q mutant”, “R179Q mutant”, and “R179W mutant”, respectively, and they are collectively referred to as “FGF23 mutants”). FGF23 mutants of the present invention are not limited thereto. The existence of other mutant proteins having the same function is predicted, and such mutants having the same function can be obtained by appropriately altering the amino acid sequence of a protein. Therefore, proteins comprising a Na/Pi cotransport function and comprising an amino acid sequence in which one or more amino acids of the amino acid sequence of SEQ ID NO: 6 are deleted, substituted or added are also included in the proteins of the present invention. An altered amino acid sequence of a protein can be obtained by well known means such as site specific mutagenes is, in which the nucleotide sequence of the DNA encoding the protein is altered, and then the DNA whose nucleotide sequence has been altered is expressed. Whether the obtained protein will regulate type II Na/Pi cotransporter expression can be confirmed by analysis methods described in the EXAMPLES.

Herein, examples of “regulation” described above was represented by “suppression”, “inhibition”, and such having a negative influence, but “regulation” in this description is not limited to negative regulation. They can also include positive regulation such as “promotion”. Therefore, an FGF23 mutant protein that may promote type II Na/Pi cotransporter expression, such as a dominant negative mutant of FGF23, can be included in the present invention.

Regulatory factors that show negative regulation decrease the expression of type II Na/Pi cotransporters, and thereby suppress the reabsorption of Pi in kidneys, and lower the blood Pi concentration. Therefore, the former can be utilized as preventive or therapeutic agents for diseases accompanying, or having the tendency of accompanying hyperphosphatemia. Hyperphosphatemia generally develops as a result of decrease in PO₄ excretion from kidneys. Progressed kidney failure (GFR less than 20 mL/minute) causes decreased excretion that is sufficient to cause increase of plasma PO₄. Even if kidney failure is absent, disorder of PO₄ excretion by kidneys may occur in the case of pseudohypoparathyroidism or hypoparathyroidism. Hyperphosphatemia may develop due to overdose of oral PO₄, and sometimes from the overuse of enema containing phosphate salts. Hyperphosphatemia may also occur as a result of transfer of intracellular PO₄ to outside cells. This occurs frequently in diabetic ketoacidosis (regardless of PO₄ loss from the entire body), contusion, non-traumatic rhabdomyolysis, and in systemic infection and tumor lysis syndrome. Hyperphosphatemia plays a critical role in secondary hyperparathyroidism, and in renal osteodystrophy patients on long-term dialysis.

On the other hand, regulatory factors showing positive regulation promote the expression of type II Na/Pi cotransporters, and thereby raise the blood Pi concentration. Therefore, regulatory factors that show positive regulation can be used for prevention and therapy of diseases and conditions that are prone to low blood Pi concentration. Herein, diseases and conditions prone to low blood Pi concentration include osteomalacia, hyperparathyroidism, abnormal vitamin D metabolism, malabsorption syndrome, fasting, glucosuria, and chronic alcoholism. Therefore, by using regulatory factors that positively regulate the expression of type II Na/Pi cotransporters against these diseases and conditions, decrease of blood phosphate concentration can be prevented or overcome. Furthermore, hypophosphatemia can occur due to complications or side effects due to therapy. Such treatments that may accompany hypophosphatemia are kidney transplantation, glucagon administration, and glucose administration. Therefore, by using regulatory factors that positively regulate expression of type I Na/Pi cotransporters during such treatments, complications and side effects such as decrease of blood phosphate concentration can be prevented.

The present invention provides genes encoding FGF23 and mutants thereof as the above-mentioned type II Na/Pi cotransporter expression modulators. DNAs encoding FGF23 are for example the DNA of SEQ ID NO: 5, which is human FGF23 cDNA. Furthermore, sequences encoding the FGF23 mutants can be produced by modifying the DNAs encoding FGF23 (for example, the DNA of SEQ ID NO: 5).

FGF23 cDNA can be prepared by methods well known to those skilled in the art. For example, it can be prepared by constructing a cDNA library from cells expressing FGF23, and performing hybridization using a part of the sequence of FGF23 CDNA (SEQ ID NO: 5) as a probe. The CDNA library can be prepared, for example, by the method described in the literature (Sambrook, J. etal. , Molecular Cloning, Cold Spring Harbor Laboratory Press (1989)), or a commercially available DNA library may be used. Furthermore, the library can also be prepared by preparing RNAs from cells expressing FGF23, and after synthesizing cDNAs using reverse transcriptase, synthesizing oligoDNAs based on the FGF23 cDNA sequence (SEQ ID NO: 5), and then performing PCR reactions using these as primers to amplify the cDNAs encoding the polypeptide of this invention.

Modification of the above-mentioned FGF23 cDNA can be accomplished by those skilled in the art using generally performed DNA mutagenesis techniques. For example, FGF-23 mutants such as R176Q, R179Q, and R179W can be produced using the above-mentioned FGF-23 cDNA (the DNA of SEQ ID NO: 5) as the template. These were constructed according to a mutagenesis method using PCR. The nucleotide sequences of primers for mutagenesis are the following.

| | |
|---|---|
| 5′-CACggCAgCACACCCggAgC-3′ | (SEQ ID NO: 9) |
| 5′-CACggCggCACACCCAgAgC-3′ | (SEQ ID NO: 10) |
| 5′-CACggCggCACACCTggAgC-3′ | (SEQ ID NO: 11) |
| 5′-CACggCAgCACACCCAgAgC-3′ | (SEQ ID NO: 12) |
| 5′-CACggCAgCACACCTggAgC-3′ | (SEQ ID NO: 13) |

Mutagenesis can be carried out by a method comprising 3 steps which involve producing a partial fragment for mutagenesis in the first PCR, then preparing a full length mutant containing a mutation as a template in the second PCR, and finally obtaining a complete mutant from the third PCR. Specifically, the first PCR is performed using the above-mentioned specific mutant primers (SEQ ID NOS: 9, 10, 11, 12, and 13) , and a specific reverse PCR primer (50 μM, SEQ ID NO: 8), the PCR products are separated by electrophoresis, and fragments of the desired length are collected. Next, a second PCR reaction is performed using the collected fragments (mutant partial sequence) to construct a template for the full length of the mutant. Subsequently, the third PCR reaction is performed using a specific forward PCR primer (SEQ ID NO: 7) , and a specific reverse PCR primer (SEQ ID NO: 8) on the second PCR reaction solution. The ultimately obtained amplification products of the PCR reactions are confirmed by electrophoresis, and the specifically amplified band at approximately 750 bp is collected. The DNAs encoding the FGF23 mutant can be obtained through this series of manipulations. Herein, FGF-23 mutants R176Q, R179Q, and R179W were specially noted for description, but other mutants can be constructed easily by those skilled in the art by referring to this description.

DNAs encoding FGF23 or mutants thereof used as the above-mentioned type II Na/Pi cotransporter expression modulator can be used as it is, or it can be used after incorporation into a vector. Such incorporation into a vector is useful for maintenance of the DNAs encoding FGF23 or mutants thereof in host cells, and for expression of FGF23 or mutants thereof. Particularly, when using a mammalian vector, it can be used for transfection of FGF23 or mutants thereof into humans. Applications to preventive and therapeutic agents for hyperphosphatemia and hypophosphatemia can be expected.

Specific examples of the above-mentioned vector, when using E. coli as a host, include M13 vectors, pUC vectors, pBR322, pBluescript, pCR-Script, pGEM-T, pDIRECT, and pT7. In order to express the proteins of this invention in E. coli, E. coli preferably carries a promoter that allows efficient expression, for example, lacZ promoter (Ward, etal., Nature (1989) 341, 544-546; and FASEBJ. (1992) 6, 2422-2427), araB promoter (Better et al., Science (1988) 240, 1041-1043) , or T7 promoter. Besides the above-mentioned vectors such expression vectors include pGEX-5X-1 (Pharmacia), “QIAexpress system” (QIAGEN), pEGFP, or pET (in this case, BL21 expressing T7 RNA polymerase is preferred as a host).

For expression in other cells, vectors must be selected according to the cells of interest. Examples include expression vectors derived from mammals (for example, pcDNA3 (Invitrogen) pEGF-BOS (Nucleic Acids. Res. 1990, 18(17), p5322), pEF, and pCDM8); expression vectors derived from insect cells (for example, “Bac-to-BAC baculovirus expression system” (GIBCO BRL), and pBacPAK8); expression vectors derived from plants (for example, pMH1 and pMH2); expression vector derived from animal viruses (for example, pHSV, pMV, pAdexLcw) ; expression vectors derived from retroviruses (for example, pZIPneo); expression vectors derived from yeast (for example, “Pichia Expression Kit” (Invitrogen), pNVll, and SP-Q01); and expression vectors derived from Bacillus subtilis (for example, pPL608, and pKTH50).

When the objective is expression in animal cells such as CHO cells, COS cells, or NIH3T3 cells, promoters necessary for expression within the cell, for example, SV40 promoter (Mulligan et al., Nature (1979) 277, 108) , MMLV-LTR promoter, EF1α promoter (Mizushima et al., Nucleic Acids Res. (1990) 18, 5322), CMV promoter must be present, and it is even more preferable if genes for selecting transformation into cells (for example, drug-resistance genes that enable differentiation by pharmaceutical agents (neomycin, G418, etc.)) are present. Examples of vectors having such characteristics include pMAM, pDR2, PBK-RSV, pBK-CMV, pOPRSV, and pOP13.

The present invention provides transformants which have been transformed by the above-mentioned genes of FGF23 or mutants thereof, or vectors carrying these genes. These transformants can be utilized mainly for the purpose of producing FGF23 or mutants thereof which are the Na/Pi cotransporter expression regulatory factors of this invention, and for the purpose of amplifying DNAs encoding FGF23 or mutants thereof.

There are no particular limitations on the host cells to which the vectors of this invention are transfected. For example, prokaryotic cells such as E. coli as well as eukaryotic cells such as animal cells maybe used. When using eukaryotic cells, for example, animal cells, plant cells, and fungal cells may be used as hosts. Exemplary animal cells include mammalian cells, such as CHO (J. Exp. Med. (1995) 108, 945), COS, 3T3, myeloma, BHK (baby hamster kidney), HeLa, Vero; amphibian cells, such as Xenopus oocytes (Valle, et al., Nature (1981) 291, 358-340); and insect cells, such as Sf9, Sf21, and Tn5. As CHO cells, dhfr-CHO in particular, which is a CHO cell whose DHFR gene has been deleted (Proc. Natl. Acad. Sci. USA (1980) 77, 4216-4220) and CHO K-1 (Proc. Natl. Acad. Sci. USA (1968) 60, 1275) can be preferably used.

The method of transfection of host cells with vectors can be appropriately selected depending on the type of cells, and may be performed by any of the methods such as infection of cells with virus particles in the case of virus vectors, biochemical methods, such as calcium phosphate method, DEAE dextran method, cationic liposome DOTAP (Boehringer Mannheim), and lipofection method, and physical methods such as electroporation method.

The present invention also provides pharmaceutical compositions, especially therapeutic agents for hyperphosphatemia, containing any one of FGF23 or mutants thereof, which are type II Na/Pi cotransporter expression regulatory factors, DNAs encoding FGF23 or mutants thereof, and the vectors carrying these DNAs. The type II Na/Pi cotransporter expression regulatory factors of the present invention regulate the expression of type II Na/Pi cotransporters, and therefore they can regulate the blood phosphorus concentration. As described above, since regulation includes both positive and negative actions, factors that negatively regulate type II Na/Pi cotransporter expression can be used as preventive and therapeutic agents for hyperphosphatemia, or as preventive and therapeutic agents for other diseases caused by hyperphosphatemia, such as inhibitors of renal failure advancement. On the other hand, factors that positively regulate type II Na/Pi cotransporter expression can be used as preventive and therapeutic agents for hypophosphatemia, or as preventive and therapeutic agents for other diseases resulting from hypophosphatemia. Such pharmaceutical compositions can be used as pharmaceutical agents for mice, rats, guinea pigs, rabbits, chickens, cats, dogs, sheep, pigs, cattle, monkeys, baboon, chimpanzee, and such, besides humans.

Other than direct administration, the above-mentioned pharmaceutical compositions can be formulated into dosage forms by conventional preparation methods and then administered to patients. For example, they can be used orally as tablets, capsules, elixirs, or microcapsules, or parenterally in the form of an injection of sterile solutions or suspensions with water or other pharmaceutically acceptable fluid. For example, the pharmaceutical composition may be formulated into the unit dosage form required by generally accepted preparation procedures by appropriately combining and mixing with pharmaceutically acceptable carriers or vehicles, more specifically, with sterilized water or physiological saline, vegetable oil, emulsifiers, suspensions, surfactants, stabilizers, flavors, fillers, preservatives, binders, and such.

Furthermore, when using the DNA of the present invention as a pharmaceutical composition, the DNA of this invention is incorporated into a vector guaranteed to express the DNA in a living body as described above, and this can be transferred into a living body, for example, by the retrovirus method, liposome method, cationic liposome method, adenovirus method, etc. This enables gene therapy against diseases that develop due to rise and fall of blood phosphorus concentration. Ex vivo methods and in vivo methods can be used for administration into a living body.

The present invention provides methods of screening for substances that interact with FGF23 or FGF23 mutants, which are type II Na/Pi cotransporter expression regulatory factor. The screening methods of the present invention comprise the steps of (A) allowing a test substance to act on a type II Na/Pi cotransporter expression regulatory factor, and (B) analyzing the presence or absence of interaction between the test compound and the type II Na/Pi cotransporter expression regulatory factor.

Especially preferable type II Na/Pi cotransporter expression regulatory factors used for screening are those that can regulate the expression of type II Na/Pi cotransporters, and wild-type FGF23 (SEQ ID NO: 6) and the above-mentioned FGF23 mutants (FGF23R176Q, FGF23R179Q, and FGF23R179W, etc.) can be used.

There are no particular limitations on the test substance, and examples include natural or synthetic compounds, various organic compounds, combinatorial libraries, natural or synthetic sugars, nucleic acids, proteins, peptides, expression products of gene libraries, cell extracts, or microbial components. A fragment of FGF23 is an example of proteins and peptides that are test samples, while an antibody against FGF23 may be another example. Examples of nucleic acids include aptamer RNA that may act on FGF23, and ribozymes and siRNA that act on FGF23 mRNA. Furthermore, SU5402, which is a tyrosine kinase inhibitor of FGFR, is known to inhibit the activity of FGF23, and this SU5402 can be employed as a test substance of this screening. Also, since FGF23 is known to bind with high affinity towards FGF receptor (FGFR)-3c expressed mainly in opossum kidney (OK) cells (Yamahita T. etal., J. Biol. Chem., In press, 2002, supra), this FGFR-3c itself or fragments thereof can be used as the test substances of the present screening.

Variety of methods exist for allowing the type II Na/Pi cotransporter expression regulatory factors to act on test substances, and these methods depend on the forms of type II Na/Pi cotransporter expression regulatory factors and the types of test substances. For example, when the test substance is an expression library plasmid DNA, it is transfected into cells expressing a type II Na/Pi cotransporter expression regulatory factor. Compounds, proteins, peptides and such can be directly allowed to act on a type II Na/Pi cotransporter expression regulatory factor, or otherwise, they can be added to a culture (liquid or solid such as agar) of cells expressing the factor. When the test substance is a protein or peptide, a DNA encoding the test substance and a type II Na/Pi cotransporter expression regulatory factor may be transferred into a cell, and both the test substance and the factor may be contacted to each other within the cell.

After allowing to act on the test substance and type II Na/Pi cotransporter expression regulatory factor, the presence or absence of interaction between the two is analyzed. Analysis of this interaction can be performed, for example, by the two-hybrid method or immunoprecipitation method. The two-hybrid method can be used when the test substance is a protein or peptide. Furthermore, the two-hybrid method can be performed using a commercially available kit. When FGF23 is precipitated using an antibody against FGF23 in immunoprecipitation, a test substance that precipitated together is considered to interact with the factor. Furthermore, fluorescence resonance energy transfer (FRET) can be used to detect interactions in a cell. In FRET, interactions between proteins and phenomena at the molecular level occurring in a cell can be detected utilizing transfer of excitation energy from a particular fluorescent molecule to another fluorescent molecule. Substances selected to have the possibility of interacting with type II Na/Pi cotransporter expression regulatory factors such as FGF23 may influence the action of FGF23 and such factors, and type II Na/Pi cotransporter expression regulating activity. Whether such substances will influence type II Na/Pi cotransporter expression regulating activity of FGF23 and such factors can be investigated by performing the various analysis methods indicated in the EXAMPLES in the presence of the selected test compounds. Furthermore, substances determined to influence type II Na/Pi cotransporter expression regulating activity of FGF23 and such factors can influence type II Na/Pi cotransporter expression through action on FGF23, and can regulate blood phosphate concentration. Particularly, the factors such as FGF23R176Q, FGF23R179Q, FGF23R179W are not cleaved by proteases, and continuously act in a suppressive manner against type II Na/Pi cotransporter expression, and, as a result, cause autosomal dominant hypophosphatemic rickets (ADHR). Therefore, if substances that act in a suppressive manner on FGF23 can be found using this screening method, they can be applied to therapeutic agents for ADHR and such diseases caused by mutations in the FGF23 gene. Furthermore, tumor-induced osteomalacia (TIO) is known as a disease that exhibits hypophosphatemia and abnormal vitamin D metabolism. In this disease, excess secretion of FGF23 from the tumor has been reported (previously described in Shimada T. et al., Proc. Natl. Acad. Sci. USA 98:6494-6499, 2001). Therefore, substances that interact in a suppressive manner on FGF23 may be used as therapeutic agents for diseases accompanying FGF23 hyperexpression. or lead compounds therefore.

### EXAMPLE 1

### Isolation of Human Type IIc Na/Pᵢ Cotransporter cDNA

The present inventors found an expressed sequence tag (EST) showing nucleotide sequence similarity to human type IIa Na/Pᵢ cotransporter by EST database searches. cDNA for the EST (GenBank™/EBI/DDBJ Accession No. AI792826) was obtained using integrated and molecular analysis of genomes and their expression (IMAGE). The ˜0.8-kb SacI fragment was excised from human cDNA (IMAGE cDNA clone 1535299) , and labeled with ³²P using the MegaPrime DNA labeling system, dCTP (Amersham Biosciences) for use as a probe to screen a human kidney 5′-Stretch Plus CDNA library (CLONTECH) Screening of the cDNA library and isolation of positive plaques were performed as described previously (Biochem. J. 305:81-85; and J. Biol. Chem. 273:28568-28575).

The human type IIc Na/Pᵢ cotransporter fragment (corresponding to nucleotides 89 to 600 of the nucleotide sequence) was used to isolate a rat type IIc Na/Pᵢ cotransporter cDNA. The oligo(dT)-primed cDNA library was prepared from rat kidney poly (A) RNA using the Superscript Choice system (Invitrogen) (J. Biol. Chem. 274:19745-19751). The synthesized cDNA was ligated to λZIPLOX EcoRI arms (Invitrogen). Screening of the cDNA library and isolation of the positive plaques were performed according to the method described previously (J. Biol. Chem. 274:19745-19751).

The human type IIc Na/Pᵢ cotransporter cDNA was 2020 bp length with 1,797 bp of open reading frame. The length of predicted amino acid sequence was 599 amino acids. Hydropathy analysis of the predicted amino acid sequence revealed the presence of eight putative transmembrane domains. The extracellular segments of human type IIc cotransporter comprised four putative N-linked glycosylation sites. Potential intracellular phosphorylation sites for protein kinase C was detected at residues 24, 152, 481, and 581 (FIG. 1a).

Amino acids in the transmembrane regions were especially well conserved among the three isoforms as shown in FIG. 1a. Amino acid comparisons revealed that the newly identified protein (type IIc) was 36% to 38% homologous to Na/Pᵢ cotransporters identified in human type IIa and type IIb amino acid sequences, respectively (Proc. Natl. Acad. Sci. USA. 90:5979-5983; and Am. J. Physiol. 276:F72-F78). Overall homology to types I and III Na/Pᵢ cotransporters was ˜10% (Biochem. J. 305:81-85; and Cell Growth & Differ. 1:119-127). The highest degrees of homology were detected in regions that have been suggested to be the transmembrane domains. The most striking difference in the newly identified protein compared with other type II Na/Pᵢ cotransporters was found in the C-terminal region comprising clusters of cysteine residues. A similar clustering of cysteine residues was also present in the type IIb Na/Pᵢ cotransporters of human, mouse, and flounder kidneys.

### EXAMPLE 2

### Tissue Distribution of Type IIc Na/Pᵢ Cotransporter

The expression of type IIc mRNA was analyzed by Northern blotting using poly(A)⁺ RNA from various human tissues and rat tissues (FIG. 1b and c). Using the novel type IIc CDNA as a probe, a strong signal was observed at about 2.4 kb only in the kidneys. No signals were detected in the brain, heart, skeletal muscle, thymus, spleen, lung, or peripheral blood leukocytes. In addition, the expression of the type IIc mRNA was significantly higher in weaning animals (22 day-old) compared with those in adults (60 day-old) (FIG. 1d). The levels of type IIc mRNA were lowest in suckling animals.

### EXAMPLE 3

### Functional Analysis of Type IIc Na/Pi Cotransporter

The functional characteristics of human type IIc Na/Pi cotransporter was analyzed in Xenopus oocytes. Expression in Xenopus oocytes was performed according to the previous report (J. Biol. Chem. 273:28568-28575, and J. Biol. Chem. 274:19745-19751). Specifically, cRNAs obtained by in vitro transcription, using T7 RNA polymerase, of the human type IIc cDNA (hNPIIC) and rat type IIa (NaPi-2) in plasmid pBluescript SK⁻ (Stratagene) were linearized with XbaI as described previously. This cRNA (25 ng) was injected into each oocyte. Two to three days after injection, Pi uptake was measured. The uptake measurement was carried out as reported previously (Biochem. J. 305:81-85, and J. Biol. Chem. 273:28568-28575). More specifically, a group of oocytes comprising six to eight cells was incubated in 500 μL of a standard uptake solution (100 mM NaCl, 2 mM KCl, 1 mM CaCl₂, 1 mM MgCl₂, 10 mM HEPES, and 5 mM Tris, pH 7.4) , or in 500 μL of an Na⁺-free uptake solution (a solution in which NaCl of the standard uptake solution is replaced with choline chloride containing a 0.1 μCi radio-labeled compound).

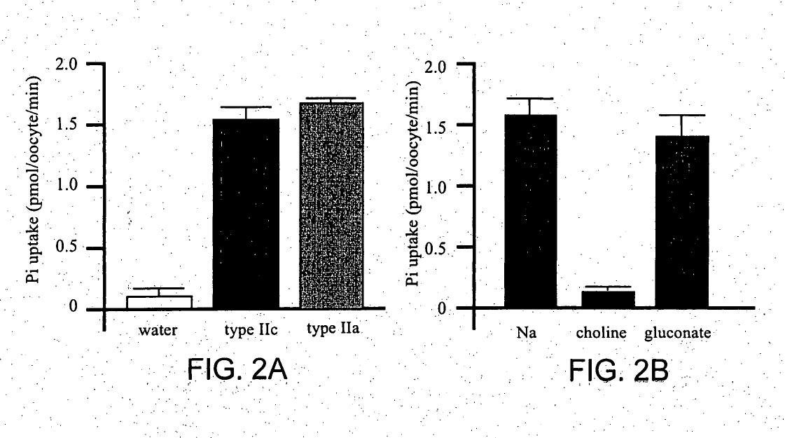

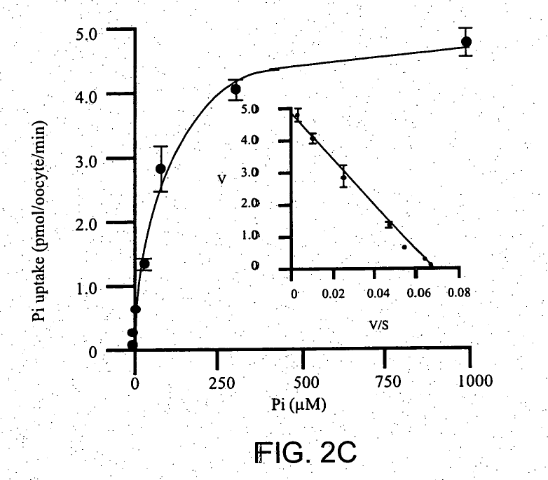

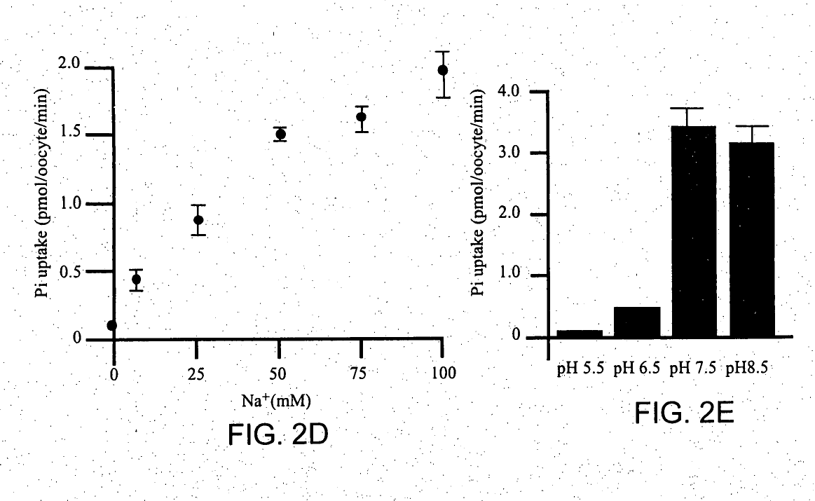

As shown in FIG. 2, Xenopus oocytes carrying a human type IIc Na/Pi cotransporter due to microinjection showed remarkable increase in Pi uptake compared to the control oocytes to which water alone had been injected (FIG. 2a). [³²P] Phosphate uptake mediated by human type IIc was dependent on Na⁺ but not Cl⁻ (FIG. 2b), and it increased in a concentration-dependent manner in the presence of Na⁺ (FIG. 2b). The uptake was saturable, and the Michaelis-Menten constant (Kₘ) for Pᵢ was 70 μM (FIG. 2c). Type IIc-mediated Na/Pᵢ uptake was stimulated by a more alkaline pH, a hallmark of proximal tubular Na/Pᵢ cotransport (FIG. 2e). The apparent K_{d} and Hill coefficient for Na interaction were K_{d}=48±9 mM and n=1.73, respectively (FIG. 2e)

### EXAMPLE 4

### Electrophysiology of IIc Na/Pi Cotransporter

Electrophysiological measurements were performed at room temperature using oocytes 3 days after cRNA injection. The oocytes were impaled with two 3 M KCl-filled electrodes with resistances of 0.5 MΩ to 2 MΩ. The electrodes were connected to a commercial two-electrode voltage clamp amplifier (CEZ 1250, Nihon Koden, Tokyo Japan) via Ag—AgCl pellet electrodes, and referenced to an Ag—AgCl pellet that was connected to the bath via a 3 M KCl-agar bridge. The voltage clamp was controlled by an analog-to-digital-to-analog interface board (Digidata 1200, Axon Instruments, Foster City, Calif.) using pCLAMP 6 software (Axon Instruments). The voltage clamp protocol was for two seconds at −80 mV to +80 mV membrane potential. The external control solution (superfusate) contained the following: 96 mM NaCl, 2 mM KCl, 1.8 mM CaCl₂, 1 mM MgCl₂, and 5 mM HEPES, pH 7.4. Phosphate was added to this solution at the indicated concentrations. The final experimental solutions were adjusted to pH 7.4. The flow rate of the superfusate was 20 ml/min, and complete exchange of the bath solution was reached within about 10 seconds.

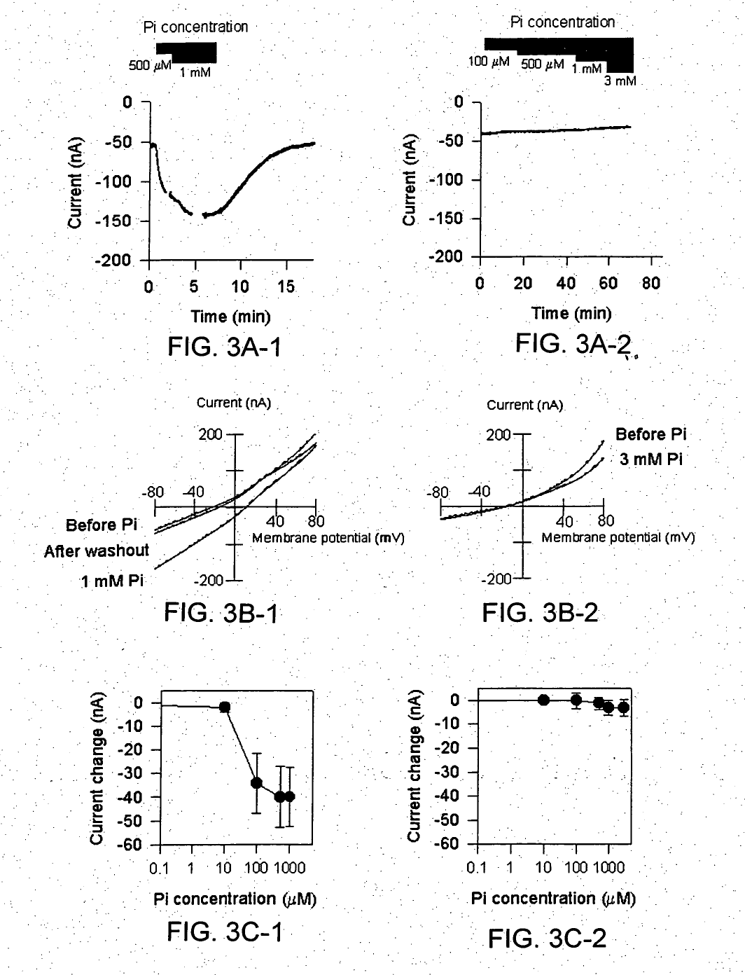

FIG. 3 shows typical time courses of currents at a membrane potential of −60 mV during the addition of Pi. Superfusion of oocytes expressing the type IIa Na/Pᵢ cotransporter with Pᵢ exhibited currents that depended on the presence of external Na⁺. Such currents were not observed, when the same protocol was applied to water or noninjected oocytes (data not shown). Washout of Pᵢ was also accompanied by a similar biphasic return to the base-line values. Reversal potential shifted from −22 mV to +16 mV during stimulation with 1 mM Pᵢ in type IIa Na/Pᵢ cotransporter-expressing oocytes. These observations suggest that the currents stimulated by 1 mM Pᵢ were Na⁺ currents. These findings confirmed the previous observation that the Na/Pᵢ cotransport by the type IIa cotransporter was electrogenic (J. Gen. Physiol. 112:1-18). In contrast, a superinfusion of oocytes expressing the type IIc cotransporter with Pᵢ (0.1 mM to 3 mM) did not exhibit the currents. These observations suggested that, unlike type IIa, Na/Pᵢ cotransport by the type IIc Na/Pᵢ cotransporter is electroneutral.

### EXAMPLE 5

### Western Blotting Analysis

The molecular weight of type IIc Na/Pᵢ cotransporter protein was determined by Western blotting analysis. Male Wister rats (3 weeks after birth) were purchased from Shizuoka Laboratory Animal Center (Shizuoka, Japan) . They were housed in plastic cages and fed standard rat chow diet (Oriental, Osaka, Japan) ad libitum for the first week. After that period, they received a diet containing 1.2% calcium and 0.6% phosphorus for 5 days. On the 6th day, the following three groups of six rats each were established: the control Pᵢ group, in which rats chronically received a diet containing 0.6% Pᵢ; the low Pᵢ group, in which rats received a diet containing a low percentage (0.02%) of Pᵢ; and the high Pᵢ group, in which the rats received a high percentage (1.2%) Pᵢ diet. After 7 days of the given diet, all of the rats were anesthetized with intraperitoneal pentobarbital, and their kidneys were removed rapidly. Brush-border membrane vesicles (BBMVs) were prepared from the removed kidneys by the Ca²⁺ precipitation method as described previously (J. Biochem. (Tokyo) 121:50-55). The levels of leucine aminopeptidase, Na⁺K⁺-ATPase, and cytochrome c oxidase were measured to assess the purity of the membranes.

Protein samples were heated at 95° C. for 5 min in sample buffer in either the presence or absence of 5% 2-mercaptoethanol, and subjected to SDS-polyacrylamide gel electrophoresis. The separated proteins were transferred electrophoretically on Hybond-P polyvinylidene difluoride (PVDF) transfer membranes (Amersham Biosciences). The membranes were treated with diluted affinity-purified anti-type IIa (1:4000) (J. Biochem. (Tokyo) 121:50-55) or type IIc (1:1000) Na/Pᵢ cotransporter antibody, and then with horseradish peroxidase-conjugated anti-rabbit IgG as the secondary antibody (Jackson ImmunoResearch Laboratories, Inc.). The signals were detected using the ECL Plus system (Amersham Biosciences) (J. Biol. Chem. 274:28845-28848).

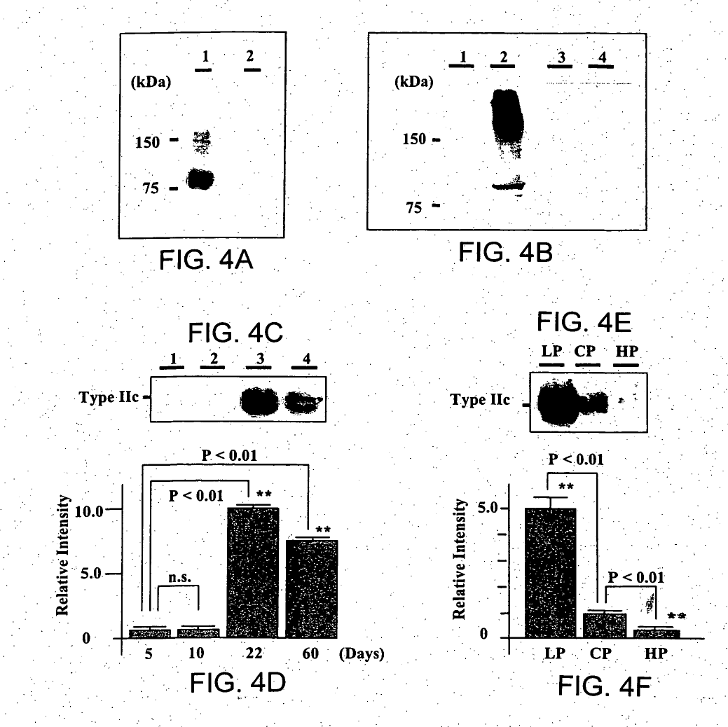

In BBMVs isolated from the rat kidney (22 day-old), the specific antibody reacted with a band of 80 to 85 kDa under reducing conditions (FIG. 4a). As measured by the presence of antigen peptides in the absorption experiments, the 80 to 85 kDa band disappeared (FIG. 4a). In addition, FLAG-fused type IIc Na/Pᵢ cotransporter in COS-7 cells was observed as 85- and 160-kDa bands using FLAG-specific monoclonal antibody (FIG. 4b) . The type IIc antibodies reacted with the 80 to 85 kDa protein band (data not shown).

In addition, the present inventors examined whether the type IIc antibodies react with type IIa Na/Pᵢ cotransporter protein. The type IIc antibodies did not react with any bands in the COS 7 cells expressing the type IIa or type IIb Na/Pᵢ cotransporters (FIG. 4b).

Next, the present inventors investigated developmental changes in rat renal type IIc protein levels (FIG. 4c). Western blot analysis demonstrated that the amount of type IIc protein in the BBMVs was highest in weaning rat, lower in adult rats, and lowest in suckling rats. In FIG. 4e, BBMVs isolated from the kidney of a rat (40 day-old) fed a diet low in Pᵢ for 7 days were prepared and used for Western blotting. The amounts of type IIc transporter protein (80 to 85-kDa band) were significantly increased (by about 5.0-fold for the 80 to 85-kDa band) compared with those in rats fed the control diet. In contrast, the high Pᵢ diet markedly suppressed the level of type IIc transporter protein.

### EXAMPLE 6

### Immunohistochemistry

Immunohistochemical analysis of the rat kidney was performed as described previously with minor modification (J. Biol. Chem. 274:28845-28848). Immunolocalization of type IIc Na/Pᵢ cotransporter protein was determined using the kidneys of weaning rats (22 day-old). For immunostaining, serial sections (5 μm) were incubated with affinity-purified anti-type IIa (1:4,000) or type IIc (1:1,000) Na/Pᵢ cotransporter antibodies overnight at 4° C. Thereafter, they were treated with Envision(+) rabbit peroxidase (Dako) for 30 min. To detect immunoreactivity against antibodies, the sections were treated with diaminobenzidine (0.8 mM).

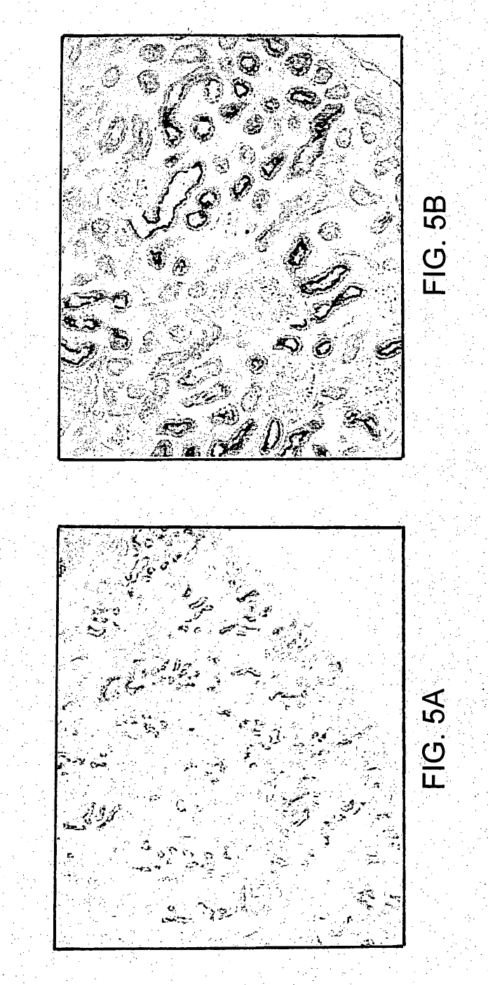

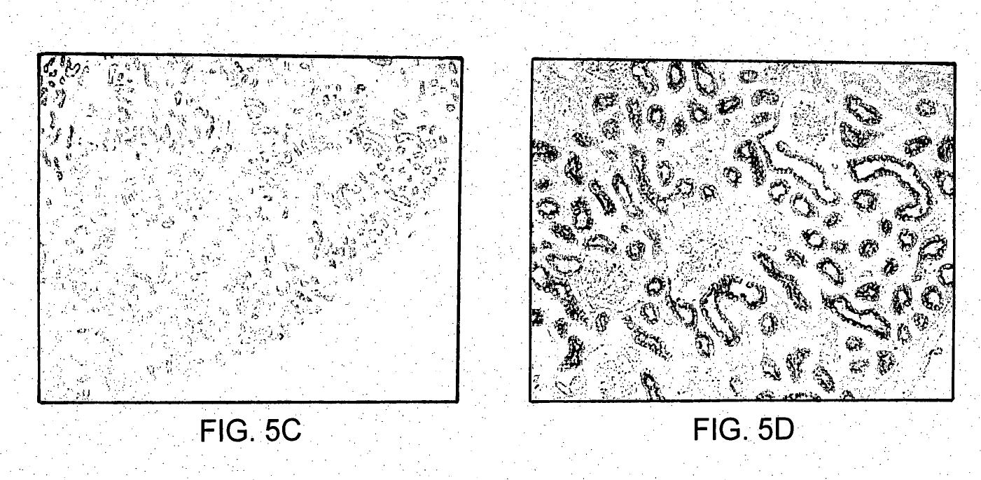

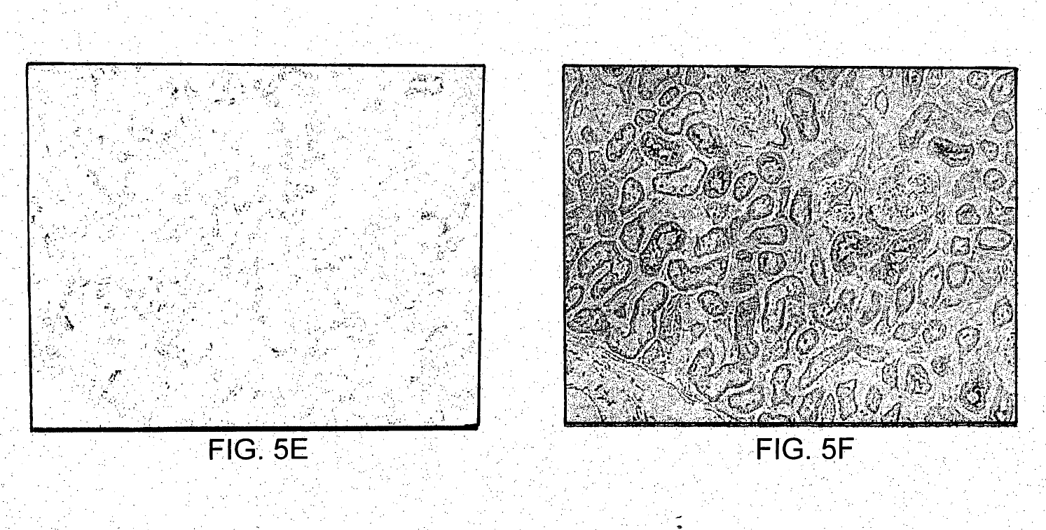

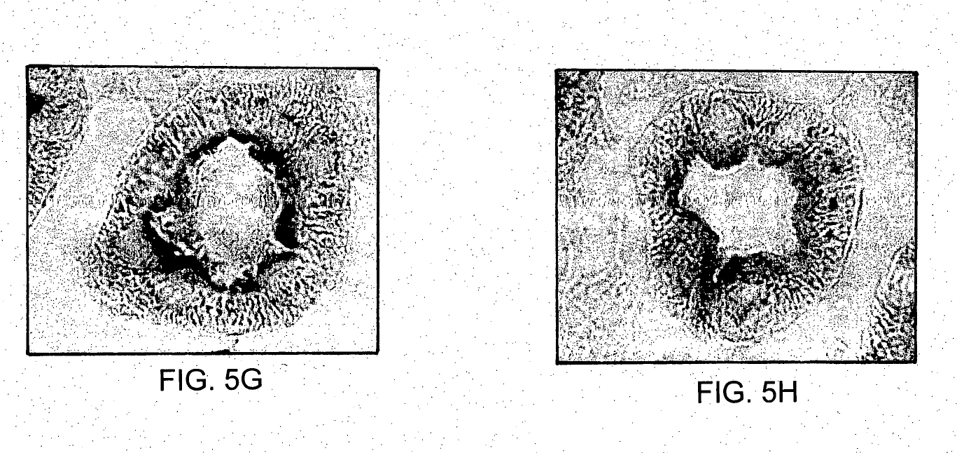

As shown in FIG. 5a and b, expression of type IIc cotransporter immunoreactive protein was detected exclusively in the superficial and juxtamedullary nephron. Similar staining was not shown using control antibodies (data not shown). The highest expression was observed in convoluted proximal tubules. At higher magnification, it was evident that type IIc transporter antibody-mediated immunoreactivity was localized in the brush border of proximal tubular cells, and was completely absent in the basolateral membrane domain (FIG. 5). Brush-border staining was slightly weaker in superficial nephrons than in juxtamedullary nephrons. In contrast, in weaning rats, type IIa antibody-related immunoreactivity was detected only in juxtamedullary nephrons (FIG. 5c and d) but not in the superficial and midcortical regions. Type IIa-related immunostaining was observed in a subapical vesicular structure, which likely belongs to the vacuolar endocytic apparatus, in weaning rat kidney (FIG. 5h). In the adult kidney (FIG. 5e and f), type IIc antibody-related immunoreactivity was detected only in juxtamedullary nephrons and not in the superficial and midcortical regions. Type IIa antibody-related immunostaining was observed in midcortical and juxtamedullary nephrons in adult rats.

### EXAMPLE 7

### Antisense Hybrid Depletion

For hybrid depletion experiments, rat kidney poly(A)⁺ RNA (5 μg/μl) was denatured at 65° C. for 5 min in solution A (50 mM NaCl and a 20 μM concentration of a 16-mer oligonucleotide complementary to rat type II phosphate transporter), and further incubated at 42° C. for 30 min (J. Biol. Chem. 267:15384-15390). 16-mer oligonucleotides complementary to rat type II phosphate transporters are as follows: sense oligonucleotide type IIa (5′-GTCCAGGGTAGAGGCC-3′ (SEQ ID NO: 14) , which is complementary to nucleotides +1004 to 1019 of rat type IIa mRNA sequence); antisense type IIa (5′-GGCCTCTACCCTGGAC-3′ (SEQ ID NO: 15) , which is complementary to nucleotides +1004 to 1019 of rat type IIa mRNA sequence); sense type IIc (5′-ATTGGCCTGGTGGACT-3′ (SEQ ID NO: 16) , which is complementary to nucleotides +134 to 149 of rat type IIc mRNA sequence); and antisense type IIc (5′-AGTCCACCAGGCCAA-3′ (SEQ ID NO: 17), which is complementary to nucleotides +134 to 149 of rat type IIc mRNA sequence) (Biochem. J. 305:81-85, and J. Biol.Chem. 273:28568-28575).

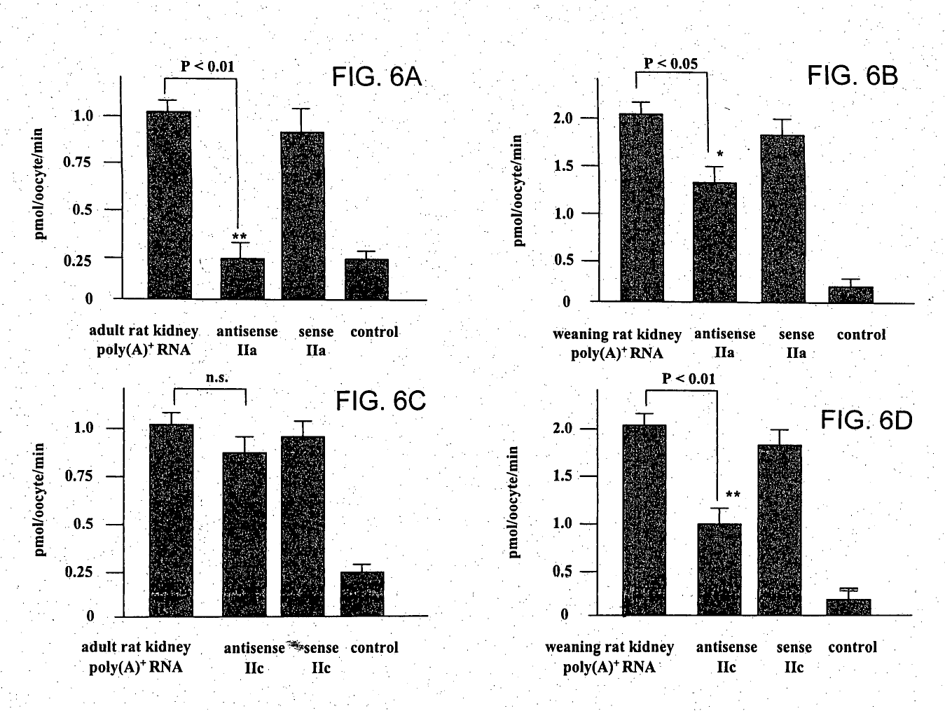

As described above, when poly (A)⁺ RNA isolated from the kidney of adult rats was treated with type IIa transporter antisense oligonucleotides of type IIa-specific mRNA, Na⁺-dependent Pᵢ uptake was completely suppressed in injected oocytes (FIG. 6a). In contrast, when poly (A)⁺ RNA isolated from the kidney of weaning rats was treated with type IIa antisense oligonucleotides, Pᵢ uptake was still detected in injected oocytes (FIG. 6b). In contrast, type IIc antisense oligonucleotides significantly suppress Pᵢ uptake in oocytes expressing poly(A)⁺ RNA from weaning rat kidney (FIG. 6d). However, similar treatment did not affect Pᵢ uptake in oocytes expressing poly(A)⁺ RNA from adult rat kidney (FIG. 6c)

### EXAMPLE 8

### Effect of FGF23 (Wild Type and R176Q Mutant) on the Levels of Plasma Calcium, Phosphate and PTH in the Rats Fed a Low Pi Diet

Male Wister rats (5 weeks after birth) were purchased from SLC (Shizuoka, Japan) for the analysis. They were housed in plastic cages and the animals were fed standard rat chow (Oriental, Osaka, Japan) ad libitum. They were fed the diet for the first week. After this period, they received a diet containing 0.6% calcium and 0.6% phosphorus for 5 days. On the 6th day, the following four groups of six rats each were established; one control Pi group, in which rats chronically received a diet containing 0.6% Pi; and three low Pi groups, in which rats received a diet containing a low percentage (0.02%) of Pi diet (Table 2). After 7 days of the given low diet, the following naked DNAs were introduced into all of rats.

The hFGF23 and the hFGF23 mutant genes were subcloned into a unique EcoRI site between CAG promoter and a 3′-flanking sequence of the rabbit β-globin gene in the pCAGGS3 expression plasmid vector (Saito H. et al., supra). The empty pCAGGS3 plasmid (provided by Dr. Miyazaki, Osaka, Japan.) was used as a mock control. The rats were intravenously injected with 12 ml of DNA solution containing 10 μg of each expression plasmid, pCGF23 (FGF23 wild type), pCGFM2 (FGF23 R176Q mutant), or empty plasmid by well-known method (Saito H. et al., supra). Four days after the naked DNA injection, blood samples were obtained from the abdominal vein under ether anesthesia. The present inventors examined the levels of plasma calcium, inorganic phosphate (Pᵢ), and PTH in obtained blood samples (Table 1).

TABLE 1
Effect of FGF23 on the levels of plasma calcium, phosphate,
vitamin D and PTH

| | | Mock | FGF23 | FGF23 (R176Q) |
|---|---|---|---|---|
| | Normal Pi | (low Pi) | (low Pi) | (Low Pi) |
| Ca | 9.8 ± 1.6 | 11.5 ± 1.3 | 8.5 ± 1.3 | 9.5 ± 1.3 |
| Pi | 8.4 ± 1.1 | 2.6 ± 0.1 | 2.2 ± 0.1 | 2.4 ± 0.1 |
| 1,25(OH)₂D₃ | 50 ± 10 | 70 ± 11 | 40 ± 8* | 20 ± 4** |
| PTH | 21.5 ± 4.5 | 12 ± 3 | 16 ± 5 | 19 ± 6 |

P < 0.05*,
P < 0.01**

In rats fed a low Pi diet, plasma calcium and 1, 25 (OH) 2D3 levels were slightly increased compared with those in rats fed a normal Pi diet. In contrast, plasma Pi and PTH levels were significantly decreased in the rats fed a low Pi diet.

Mutant FGF23 injection to the rats fed a low Pi diet did not affect the levels of plasma Pi, calcium, and PTH compared with the mock-injected control. In contrast, the levels of plasma 1, 25 (OH) 2D3 were lower in FGF23 (wild type) and FGF23 (R176Q) mutant compared with the mock-injected control.

### EXAMPLE 9

### Naked FGF23 DNA (Wild Type and R176Q Mutant) Injection Into Rats in Vivo

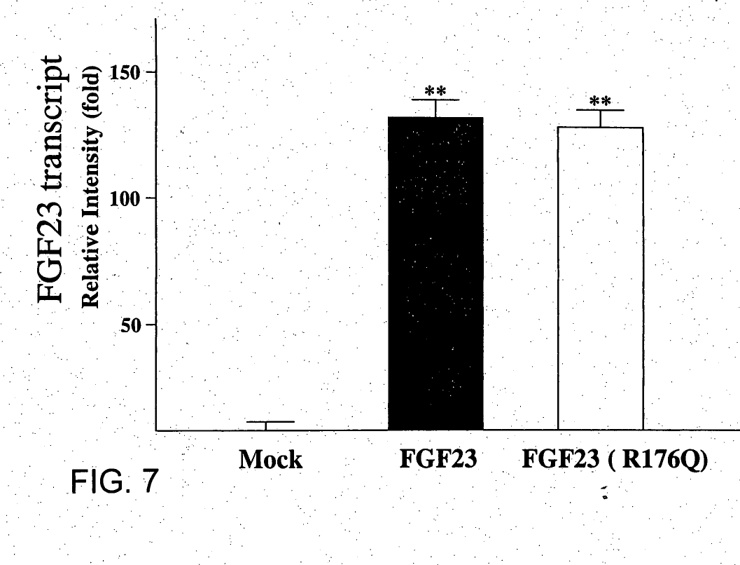

In a previous study, the present inventors investigated whether FGF23 affects renal Pi cotransport activity in vivo. A naked DNA injection (FGF23R176Q) significantly suppressed sodium-dependent Pi transport activity and 1-hydroxyvitamin D3 production in the kidney (Saito H. et al., supra). In rats injected with naked DNA (FGF23 or FGF23R176Q) , the levels of FGF23 transcripts in the liver were increased 150-fold compared with the non-treated control. In contrast, the present inventors could not detect the expression of FGF23 transcripts in the liver of the mock-injected control (FIG. 7).

Next, the present inventors determined the sodium-dependent Pi transport activity in renal BBMV isolated from rats fed a low Pi diet in Example 8 above. The Pi transport activity was measured in terms of the uptake of radiolabelled Pi by the rapid-filtration technique. After 10 μl of the vesicle suspension had been added to 90 μl of the incubation solution (containing 100 mM NaCl, 100 mM mannitol, 20 mM Hepes/Tris and 0.1 mM KH₂PO₄) , the preparation was incubated at 20° C. The measurements of Na⁺-dependent Pi uptake were performed as described previously (Katai K., Segawa H., Haga H., Morita K., Arai H., Tatsumi S., Taketani Y., Miyamoto K., Hisano S., Fukui Y., and Takeda E., Acute regulation by dietary phosphate of the sodium-dependent phosphate transporter (NaP(i)-2) in rat kidney., J. Biochem. 121:50-55, 1997; Takahashi F., MoritaK., Katai K., Segawa H., Fujioka A., Kouda T., Tatsumi S., Nii T., Taketani Y., Haga H., Hisano S., Fukui Y., Miyamoto K., and Takeda E.. Effects of dietary Pi on the renal Na⁺-dependent Pi transporter NaPi-2 in thyroparathyroidectomized rats., Biochem. J. 333: 175-181, 1998; and Taketani Y., Segawa H., Chikamori M., Morita K., Tanaka K., Kido S., Yamamoto H., Iemori Y., Tatsumi S., Tsugawa N., Okano T., Kobayashi T., Miyamoto K., and Takeda E., Regulation of type II renal Na⁺-dependent inorganic phosphate transporters by 1, 25-dihydroxyvitamin D3. identification of a vitamin D-responsive element in the human NaPi-3 gene., J. Biol. Chem. 273: 14575-14581, 1998). Transport was terminated by rapid dilution with 3 ml of an ice-cold saline. The reaction mixture was then immediately transferred to a pre-moistened filter (0.45 μm) maintained under a vacuum.

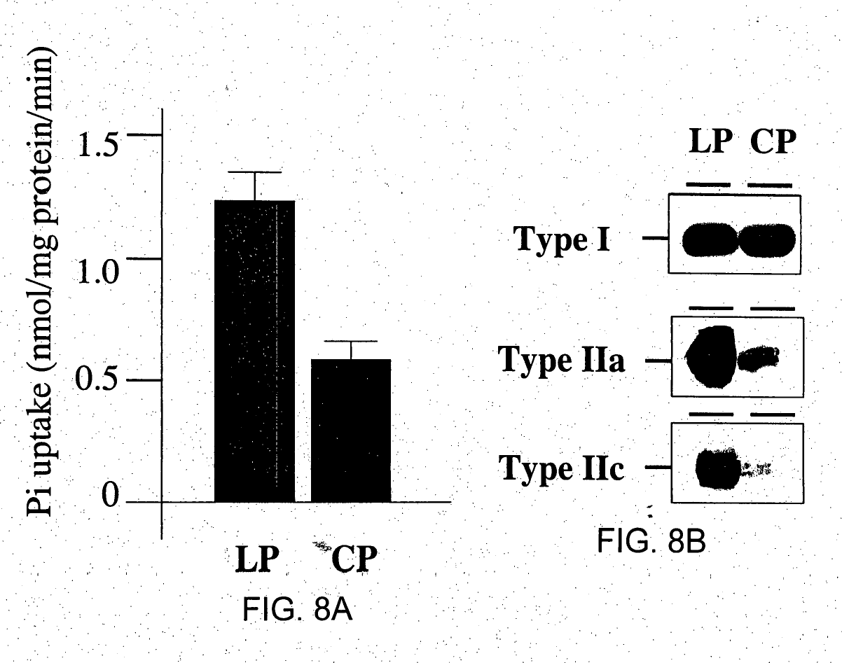

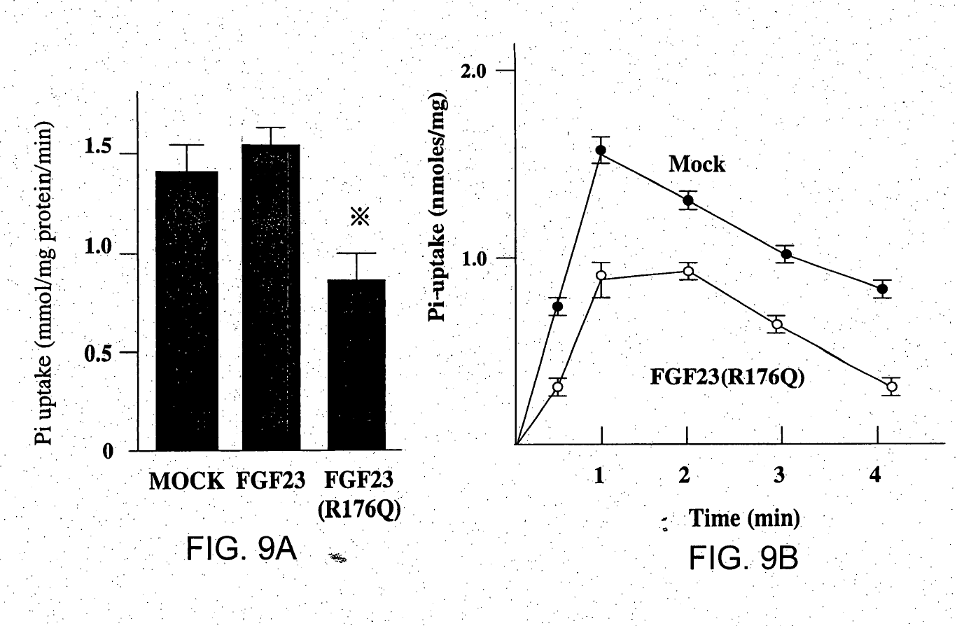

As shown in FIG. 8a, the Pi transport activity was increased in the rats fed a low Pi diet. In addition, the levels of type IIa and type IIc proteins were significantly increased in rats fed a low Pi diet compared with those fed a normal Pi diet (FIG. 8b). Four days after the administration of FGF23 (R176Q), the Pi transport activity (at 1 min) in the rats was decreased to 60% of the mock-injected control (1.46±0.2 vs 0.85±0.2) (FIG. 9). The reduction in Pi transport activity was due to a decrease in Vmax, but not Km (data not shown). In contrast, FGF23 (wild type) did not affect the activity of renal Pi transport in BBMVs (FIG. 9a).

### EXAMPLE 10

### Effect of FGF23 on the Expression of Type IIa and Type IIc Na/Pi Cotransporter in the BBMVs

The present inventors next analyzed effect of FGF23 (R176Q) on the expression of type I, type IIa and type IIc Na/Pi cotransporter in rats fed a low Pi diet.

Brush-border membrane vesicles (BBMVs) were prepared from the rat kidney by the Ca²⁺ precipitation method as described in Example 5 above. The levels of leucine aminopeptidase, Na⁺K⁺-ATPase, and cytochrome c oxidase were measured to assess the purity of the membranes. Protein samples were heated at 95° C. for 5 min in sample buffer in either the presence or absence of 5% 2-mercaptoethanol, and subjected to SDS-polyacrylamide gel electrophoresis. The separated proteins were transferred electrophoretically to Hybond-P polyvinylidene difluoride transfer membranes (Amersham Pharmacia Biotech). The membranes were treated with diluted affinity-purified anti-type IIa (1:4,000) or type IIc (1:1,000) Na/Pi cotransporter antibody, and then with horseradish peroxidase-conjugated anti-rabbit IgG as the secondary antibody (Jackson ImmunoResearch Laboratories, Inc.). The signals were detected using the ECL Plus system (Amersham Pharmacia Biotech).

Antibodies used in this Example and subsequent Examples were prepared as follows. The oligopeptide (CLALPAHNATRL) corresponding to the amino acid residues (626-637) of rat type IIa Na/Pi cotransporter and the oligopeptide (CYENPQVIASQQL) corresponding to the amino acid residues (590-601) of rat type IIc Na/Pi cotransporter were synthesized. The N-terminal cysteine residues were introduced for conjugation with keyhole limpet hemocyanine. Rabbit anti-peptide antibodies were produced as described previously . The produced antibodies were affinity purified before use.

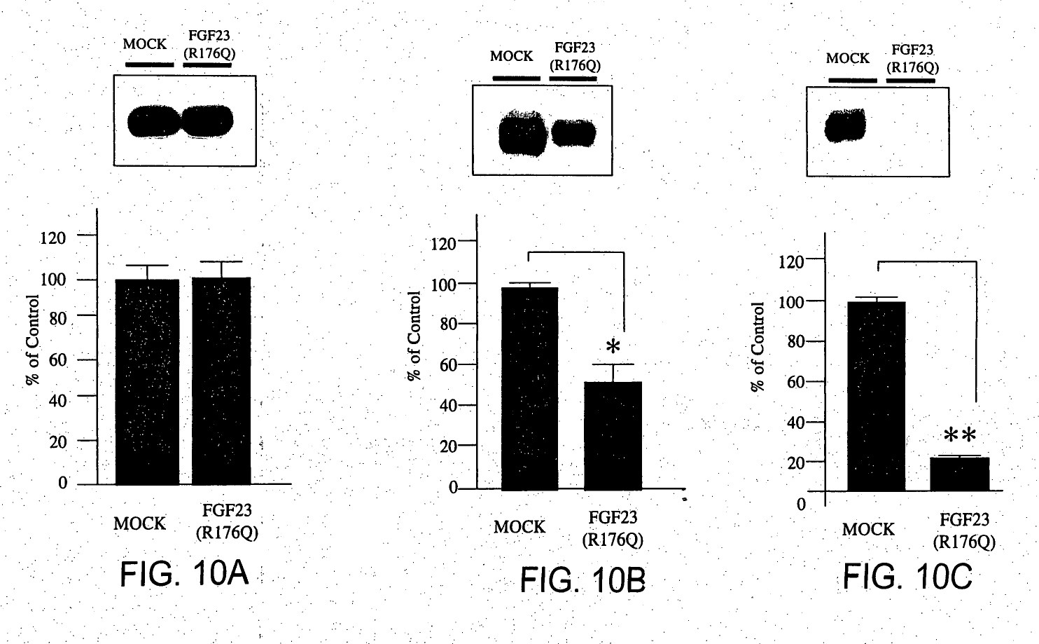

Expression levels of type IIa and type IIc Na/Pi cotransporters in FGF23(R176Q)-treated Pi-depleted rats decreased to 55% and 20% compared to that of the mock-injected control, respectively (FIG. 10b and c). On the other hand, type I Na/Pi cotransporter expression level did not change (FIG. 10a) . Therefore, FGF23 was shown to specifically affect type II Na/Pi cotransporter expression.

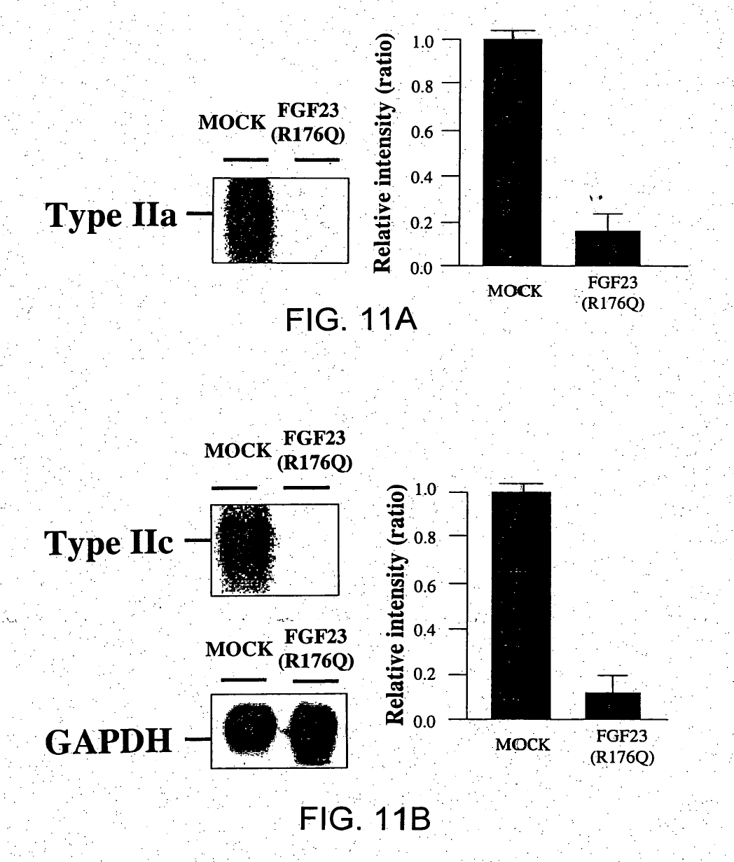

Furthermore, type IIa and type IIc transcription product levels in the renal cortex were analyzed as follows using the above-mentioned rats (FIG. 11). Liver total RNA in naked-DNA injected animals was extracted using ISOGEN (NIPPON GENE, Tokyo, Japan). Using probes specific to each of the transcription products, Northern blotting was performed. The probe used to detect rat type IIa transporter mRNA was a 1096 bp-long fragment (SEQ ID NO: 18) corresponding to nucleotides 546-1639 of the rat type IIa transporter cDNA registered as sequence ac #L13257. The probe used to detect rat type IIc transporter mRNA was a 436 bp-long fragment (SEQ ID NO: 19) corresponding to nucleotides 82-517 of the rat type IIc transporter cDNA registered as sequence ac #AB077042.

Upon measuring the expression densities of type IIa and type IIc transcription products from the signal intensity of northern blotting, they were found to be decreased by 80% and 87% compared to that of the control rat, respectively.

### EXAMPLE 11

### Immunohistochemical Analysis of Type IIa and Type IIc Na/Pi Cotransporters in Kidneys

Next, the present inventors analyzed the distribution and localization of type IIa and IIc transporters using immunohistochemistry. Immunohistochemical analysis in rat kidneys was performed according to the previous report with slight modifications.

For preparation of antibodies to type IIa and IIc transporters for use in immunostaining, the oligopeptide (CLALPAHNATRL) corresponding to the amino acid residues (626-637) of rat type IIa Na/Pi cotransporter and the oligopeptide (CYENPQVIASQQL) corresponding to the amino acid residues (590-601) of rat type IIc Na/Pi cotransporter were synthesized. The N-terminal cysteine residues were introduced for conjugation with keyhole limpet hemocyanine. Rabbit anti-peptide antibodies were produced as described previously. The produced antibodies were affinity purified and used in the following analysis.

For immunostaining, serial sections (5 μm) were incubated with anti-type IIa (1:4,000) or type IIc (1:1,000) Na/Pi cotransporter antibodies, overnight at 4° C. Thereafter, they were treated with Envision (+) rabbit peroxidase (Dako) for 30 min. To detect immunoreactivity, the sections were treated with diaminobenzidine (0.8 mM).

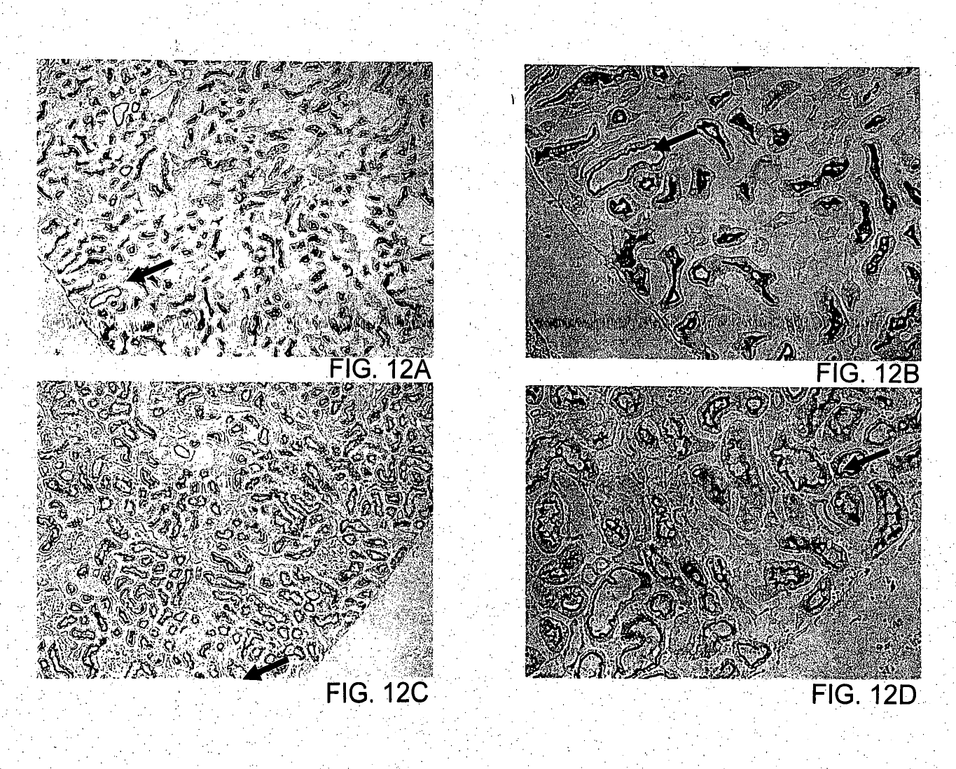

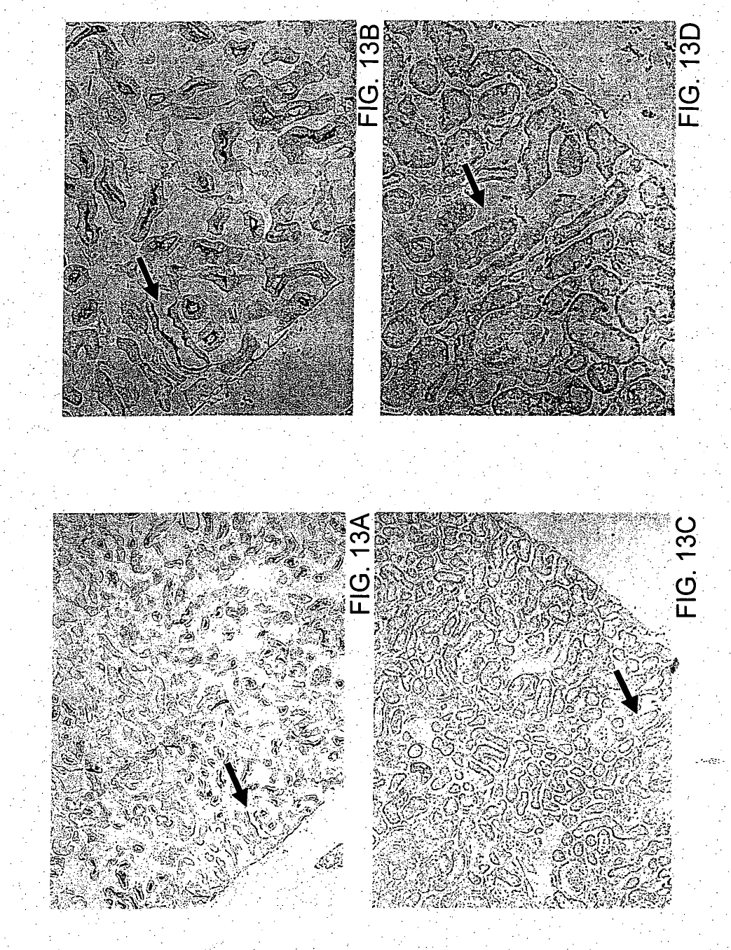

In the rats fed a normal Pi diet, type IIa and type IIc immunoreactive signals were detected in the apical membrane in midcortical nephrones (data not shown). After feeding a low Pi diet, the type IIa immunoreactive signals were markedly increased in the apical membranes of the proximal tubules (S1 , S2 and S3) in superficial and midcortical nephrons. In the mock-injected rats, the expression of type IIa and type IIc immunoreactive signals was not affected in the apical membrane of the superficial and midcortical nephrons (FIG. 12a and b, and FIG. 13e and f) . Administration of FGF23 (R176Q) into the rats fed a low Pi diet decreased the intensity of the immunoreacive signals of the type IIa transporter in the apical membranes of renal proximal tubular cells (FIG. 12c and d). In addition, FGF23 (R176Q) completely decreased the immunoreactive signals of type IIc Na/Pi cotransporter in the apical membranes of the proximal tubules in rats fed a low Pi diet (FIG. 13g and h).

### EXAMPLE 12

### Change in Serum Phosphorus and Calcium Concentrations Due to Administration of a FGF23 Mutant (FGF23R179Q)

Male Wistar rats weighing approximately 200 g were used for the experiment. The rats were individually housed in stainless cages and raised in an incubator in a breeding room under light-dark cycle conditions (8:00-20:00). They were fed a low phosphorus diet (Pi: 0.02%, Ca: 0.6%) such as that shown in Table 2 (previously described in Takahashi F. et al., Biochem. J. 333:175-181, 1998; and Levi M., Lotscher M, Sorribas V., Custer M. et al., Cellular mechanisms of acute and chronic adaptation of rat renal Pi transporter to alterations in dietary Pi., Am. J. Physiol. Renal Fluid Electrolyte Physiol. 267:F900-F908, 1994). Regarding water, tap water containing calcium and phosphorus as trace elements was avoided, and distilled water was given ad libitum. After breeding for one week, FGF23 mutant (FGF23R179Q) expression plasmid or MOCK plasmid was administered using naked DNA injection method (TransIT In vivo Gene Delivery System TaKaRa) (Niwa H., Yamamura K., Miyazaki J., Efficient selection for high-expression transfectants with a novel eukaryotic vector., Gene 108:193-200, 1991). 12 mL of DNA solution containing 10 μg of each plasmid was prepared, and this was injected into the rat-tail vein. Four days later, blood was collected under etherization, the animal was sacrificed, and immediately thereafter their kidneys and small intestine were removed.

TABLE 2

| Feed composition (/100 g) | |
|---|---|
| Concentration in feed | |
| Ca (%) | 0.6 |
| P (%) | 0.02 |
| Cornstarch | 39.7486 |
| Egg white casein | 20.0000 |
| α-modified cornstarch | 13.2000 |
| Soybean oil | 7.0000 |
| Cellulose powder | 5.0000 |
| Mineral mix | 1.5645 |
| Vitamin mix | 1.0000 |
| L-cystine | 0.3000 |
| Choline bitartrate | 0.2500 |
| Tertiary butylhydroquinone | 0.0014 |
| CaCO₃ | 1.4984 |
| KH₂PO₄ | 0.0000 |
| Sucrose | 10.4371 |
| | 100.0 |

Modified AIN-93G purified diet
Ca and P sources removed from AIN-93G-MIX

Phosphorus concentration and calcium concentration in the blood sera obtained from the FGF23R179Q-administered group and MOCK-administeredgroupweremeasuredbythe followingmethod. Blood phosphorus concentration was measured by p-methylaminophenol reduction method using Phospha C-test Kit (WAKO). Blood calcium concentration was measured by the methylxylenol blue (MXB) method using Calcium E-test Kit (WAKO).

The mean values of phosphorus concentration and calcium concentration in the serum are shown in Table 3. Serum phosphorus concentration and calcium concentration in the FGF23 mutant-administered group both showed a decreasing trend.

TABLE 3
Blood serum phosphorus and calcium concentrations

| | Phosphorus | Calcium |
|---|---|---|
| | concentration | concentration |
| | (mg/dl) | (mg/dl) |
| Mean of FGF23R179Q-administered | 4.27 ± 0.770 | 10.99 ± 1.512 |
| group (n = 6) | | |
| Mean of MOCK-administered group | 4.67 ± 0.947 | 14.54 ± 0.919 |
| (n = 4) | | |

The values are indicated as means ± standard deviation.

### EXAMPLE 13

### Effect of FGF23R179Q on Type IIa NaPi Cotransporter Carrier Protein Expression in Renal Brush Border Membrane Vesicles (BBMVs)

Renal BBMVs were prepared by the Ca2+ precipitation method (Kessler M., Acuto O., Storelli G., Murer M. et al., A modified procedure for the rapid preparation of efficiently transporting vesicles from small intestinal brush border membranes. Their use in investigating some properties of D-glucose and choline transport systems. Biol. Biochem. 162:156-159, 1987; and Minami H., Kim J. R., Tada K., Takahashi F. et al, Inhibition of glucose absorption by phlorizin affects intestinal functions in rats. Gastoenterology 105:692-697, 1993). To the removed kidneys, 30 times the organ weight of homogenate buffer (50 mM mannitol, 2 mM Tris-HCl pH 7.5) was added, and this was homogenized for two minutes using a Waring blender. Calcium chloride solution was added to this homogenate solution so that the final concentration became 10 mM, and while cooling on ice, this was stirred gently for 15 minutes. The resulting mixture was then centrifuged at 5,000 rpm for 15 minutes, the supernatant was filtered, and this filtrate was further centrifuged at 18,000 rpm for 30 minutes. A suspension buffer (300 mM mannitol, and 10 mM N-2-hydroxyethylpiperazine-N′-2-ethanesulfonic acid (Tris-HEPES) pH 7.5) was added to the resulting precipitates to prepare a suspension. After homogenizing further with a homogenizer, the homogenized product was centrifuged at 18,000 rpm for 45 minutes. The obtained precipitates were suspended in a suspension buffer using a 22G needle, and then further suspended using a 27G needle to form a BBMV preparation. Protein quantification using BCA Protein Assay Kit (PIERCE) was performed on this BBMV preparation.

To BBMVs obtained from rat kidneys of the FGF23R179Q administered group and the MOCK-administered group, equal volumes of the sample buffer (1 M Tris-HCl pH 6.8, 10% SDS, glycerol, bromophenol blue, β-mercaptoethanol) were mixed, these were heated at 95° C. for five minutes, and then immediately cooled on ice. Renal brush border membrane vessicles (20 μg/well) were separated using 10% SDS-polyacrylamide gel, and then electrotransferred to a nitrocellulose membrane, Hybond-P (Amersham Pharmacia Biotech) After blocking using 5% skim milk/1×TBST (Tris-base, NaCl pH 7.61/Tween 20) at room temperature for one hour, this was incubated with anti-type IIa NaPi cotransporter antibody (10,000 times dilution) at 4° C. for a whole day. Furthermore, this membrane was incubated at room temperature for one hour with Horse radish peroxidase (HRP)-labeled anti-rabbit IgG antibody (10,000 times dilution), then this was chemiluminesced for one minute using ECL+ plus Kit (Amersham), and then detected by exposure to X-Omat film (Kodak).

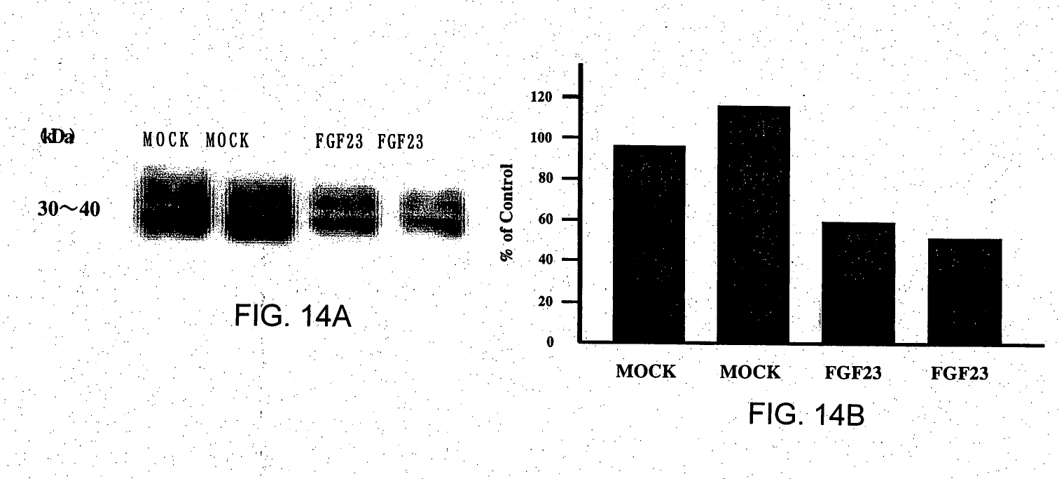

According to the above-mentioned Western blot analysis, the Type IIa antibody was shown to react with a 30 kDa to 40 kDa protein (FIG. 14A) . Comparing each group regarding this protein, expression decreased to approximately 50% in the FGF23R179Q-administered group when compared to the MOCK-administered group (FIG. 14B).

### EXAMPLE 14

### Effect of FGF23R179Q on Type IIc NaPi Cotransporter Expression in Renal Brush Border Membranes

Western blot analysis was performed by a procedure similar to that indicated in Example 13 above, except that anti-type IIc NaPi cotransporter antibody (1,000 times dilution) was used as the antibody instead of anti-type IIa NaPi cotransporter antibody.

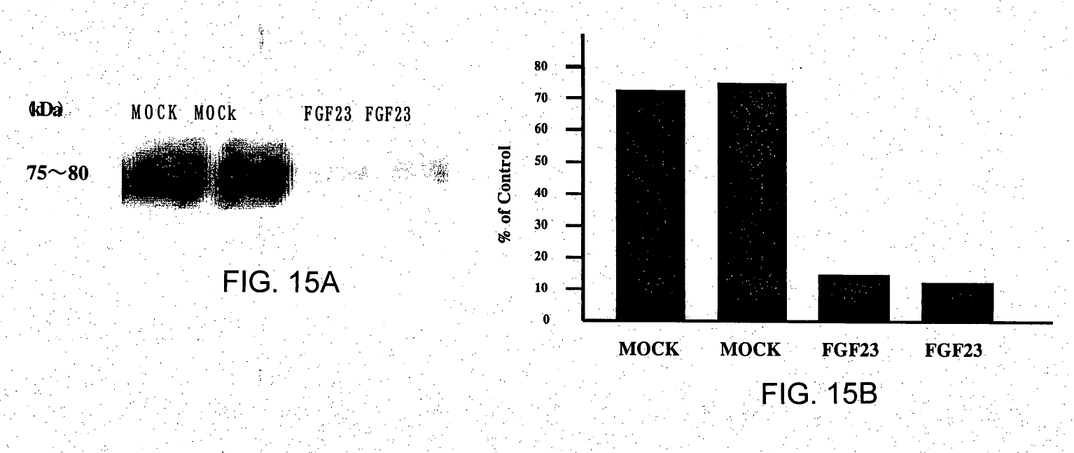

The type IIc antibody was shown to react with a 75 kDa to 80 kDa protein. Comparing each group regarding this protein, expression in the FGF23R179Q-administered group decreased to the point where it was almost undetectable (FIG. 15).

### EXAMPLE 15

### Effect of FGF23R179Q on Type I NaPi Cotransporter Expression in Renal Brush Border Membranes

Western blot analysis was performed by a procedure similar to that indicated in Example 13 above, except that anti-Type I NaPi cotransporter antibody (1,000 times dilution) was used as the antibody instead of anti-Type IIa NaPi cotransporter antibody.

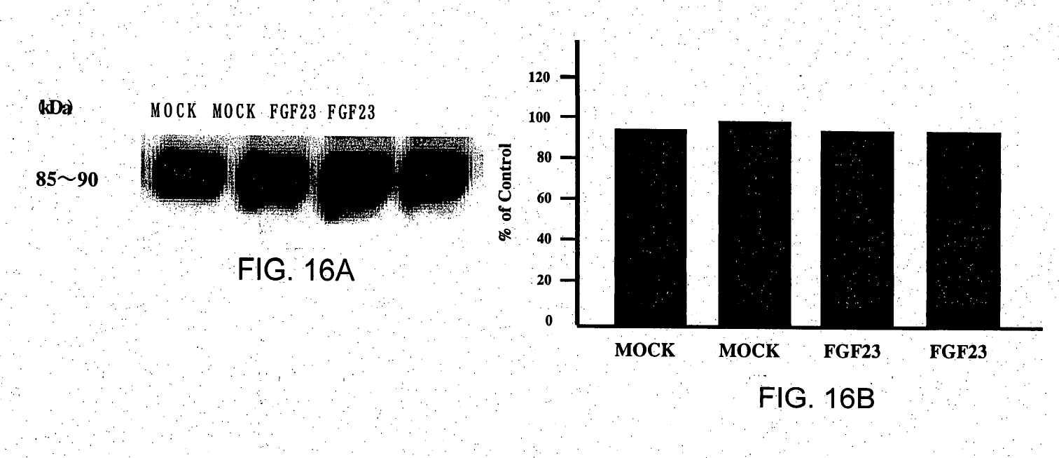

The Type I antibody was shown to react with a 85 kDa to 90 kDa protein. Comparing each group regarding this protein, a significant difference in expression level was not observed in the FGF23R179Q-administered group compared to that of the MOCK-administered group (FIG. 16).

### EXAMPLE 16

### Change in Phosphorus Transport Activity Due to FGF23R179Q Administration in the Renal and Small Intestinal Brush Border Membranes

Activity of ³²P transport to the rat renal BBMVs in the FGF23Rl79Q-administered group and MOCK-administered group was measured by the rapid membrane filtration method (previously describedinKataiK. etal., J. Biochem. 121:50-55, 1997; and Nakagawa N., Arab N., and Ghisham F. K., Characterization of the defect in the Na⁺-phosphate transporter in vitamin D-resistant hypophosphatemic mice., J. Biol. Chem. 266:13616-13620, 1991). BBMVs were ultimately suspended in a suspension buffer (300 mM mannitol, 10 mM Tris-HEPES pH 7.5). 20 μg of BBMV suspension solution, and 100 μL of ³²P solution into which 100 mM unlabeled H₂PO₄⁻ has been added (20 μCi/mL, 100 mM NaCl, 100 mM mannitol, 20 mM Hepes/Tris pH 7.5) were mixed. The mixture was added dropwise to a nitrocellulose membrane (pore size 0.45 μM) (ADVANTEC) and filtered by suction. The membrane was washed three times with 3 mL of ice-cooled physiological saline, and then radioactivity was measured bya scintillation counter. Since phosphorus transport activity reaches a saturated state in a time-dependent manner as the reaction progresses, examination was carried out in 30 seconds.

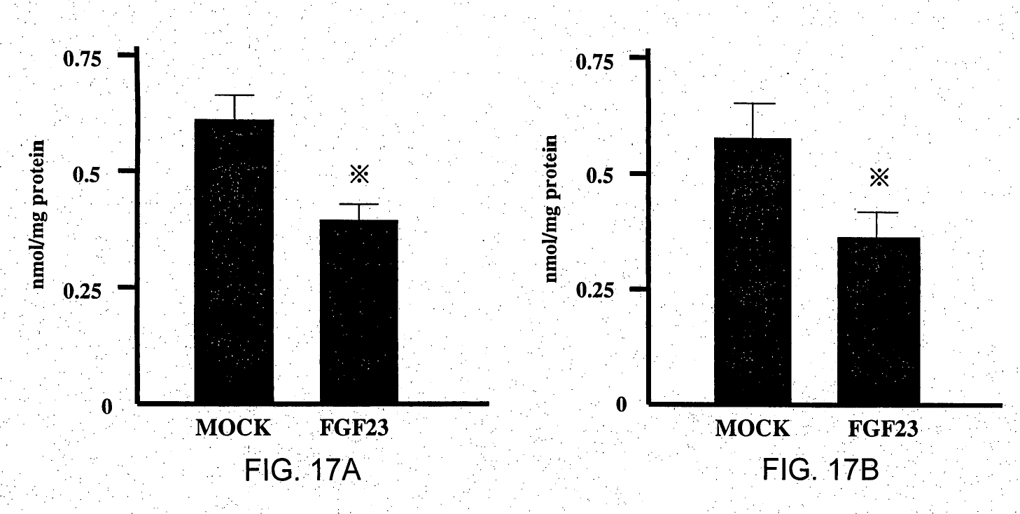

FGF23R179Q administration caused a significant decrease of phosphorus transport activity in the kidneys and small intestine (FIG. 17) (p<0.05).

### EXAMPLE 17

### Effect of FGF23Rl79Q on Type IIa NaPi Gene Expression in Kidneys

To approximately 0.5 g of rat kidneys of the FGF23Rl79Q-administered group and MOCK-administered group, 10 times that amount of ISOGEN (WAKO) was added. The kidneys were then homogenized by polytron, and further disrupted by passing through a 22G needle. For every 1 mL of ISOGEN added, 0.2 mL of chloroform was added, and the solution was mixed for approximately 15 seconds, then left to stand at room temperature for two minutes. After centrifugation at 12,000 rpm for 10 minutes at 4° C., 2.5 mL of 2-propanol was added to the supernatant, and the mixture was left to stand at room temperature for five minutes. This was then centrifuged at 12,000 rpm for 10 minutes at 4° C., 75% ethanol was added to the precipitates that formed. After centrifugation at 7,500 rpm for six minutes at 4° C., this was dried in vacuo. The obtained precipitates were dissolved in 250 μL of aqueous diethylpyrocarbonate (DPC) to yield total RNA (Chomczynski P., Sacchi N., Single-step method of RNA isolation by acid guanidinium thiocyanate-phenol-chloroform extraction. Anal. Biochem. 162:156-159, 1987).

The above-mentioned purified total RNA (20 μg) was electrophoresed through a 1.0% agarose gel, then the RNA was transferred overnight onto a nylon membrane filter, Hybond N⁺ (Amersham Pharmacia Biotech). Prehybridization was performed on this filter using Rapid-hybridization buffer (Amersham pharmacia biotech) at 65° C. for one hour, then a probe was added and this was hybridized at 65° C. for four hours. The probe was synthesized using MegaPrime DNA labeling kit (Amersham pharmacia biotech) in the presence of [α-³²P] dCTP. Rat NaPi IIa (20) and GAPDH cDNA were used as templates. Washing was carried out once with 2×SSPE (300 mM sodium chloride, 23 mM sodium dihydrogen phosphate, and 2 mM EDTA 2Na)-0.1% SDS; twice with 1×SSPE-0.1% SDS; three times with 0.2×SSPE-0.1% SDS; and twice with 0.1×SSPE-0.1% SDS. Each wash was performed for five minutes at room temperature. For the analysis, bioimage analyzer Fujix BAS 2000 (Fuji film) was used.

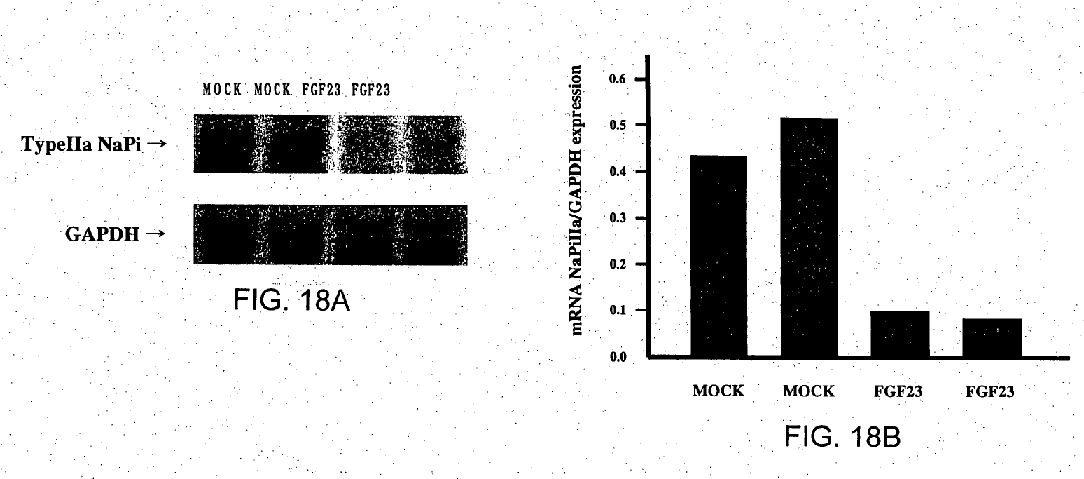

The expression level of type IIa NaPi mRNA was examined. Type IIa NaPi mRNA was a size of 2.4 kb. Upon comparison to the expression level of the MOCK-administered group, a significant decrease of expression was observed in the FGF23R179Q-administered group (FIG. 18).

### EXAMPLE 18

### Immunohistochemical Staining Analysis of Type IIa NaPi Cotransporter in Renal Brush Border Membranes

Abdominal incision was performed on rats immediately after nembutal anesthesia (1 mL/kg). 50 μL of heparin, followed by approximately 40 mL of physiological saline were perfused through the heart, immediately followed by perfusion fixation with approximately 200 mL of 4% paraformaldehyde solution. The kidneys and small intestine were removed after completion of perfusion, and were cut into an appropriate size, and then immersion fixed overnight in 4% paraformaldehyde at 4° C. Next, the immobilized kidney and small intestine pieces were immersed overnight in 20% sucrose 1×PBS at 4° C., and then freeze-embedded in OCT-Compound (SAKURA) at −80° C. The frozen section was sliced on a cryostat to a thickness of 5 μm, and was placed onto a pre-silanized slide. This was microwave treated with 1 mM citric acid monohydrate (pH 6.0) , and antigen inactivation reaction was performed. This was followed by blocking for two hours at room temperature using 5% goat serum/0.05 M TBST (Tris-base, NaCl pH 7.61/Tween 20), then this was incubated for a whole day at 4° C. with anti-rat type IIa sodium-dependent phosphate transporter antibody or type IIc sodium-dependent phosphate transporter antibody. Envision (+) rabbit peroxidase (Dako) was used as a secondary antibody, and reacted for 30 minutes. Then, this was stained by incubating at room temperature for 5 to 10 minutes with a staining solution (DAB/PBS) dehydrated using 70% (one minute), 80% (one minute) and 100% (one minute) ethanol, and xylene (one minute×3), and enclosed using Mount-quick (Daido Sangyo).

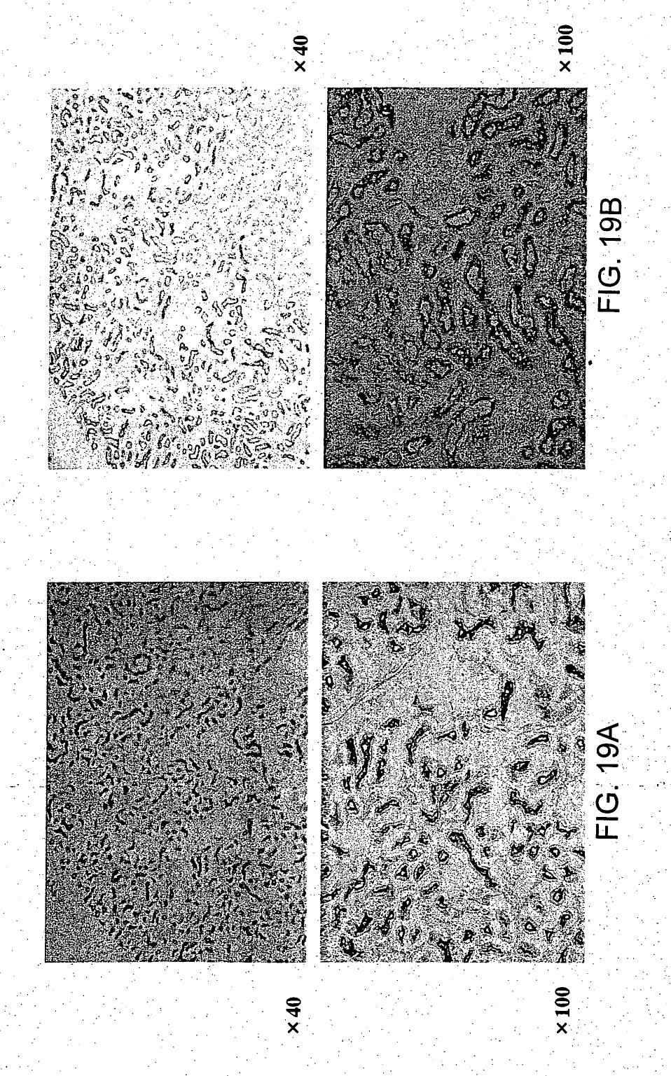

Upon immunohistochemical staining using a specific antibody of the above-mentioned type IIa NaPi cotransporter, expression in the renal proximal tubular brush border membrane due to intake of low phosphorus diet was clearly observed in the MOCK-administered group. Such expression diminished significantly in the FGF23Rl79Q-administered group (FIG. 19).

### EXAMPLE 19

### Immunohistochemical Staining Analysis of Type IIc NaPi Cotransporter in Renal Brush Border Membranes

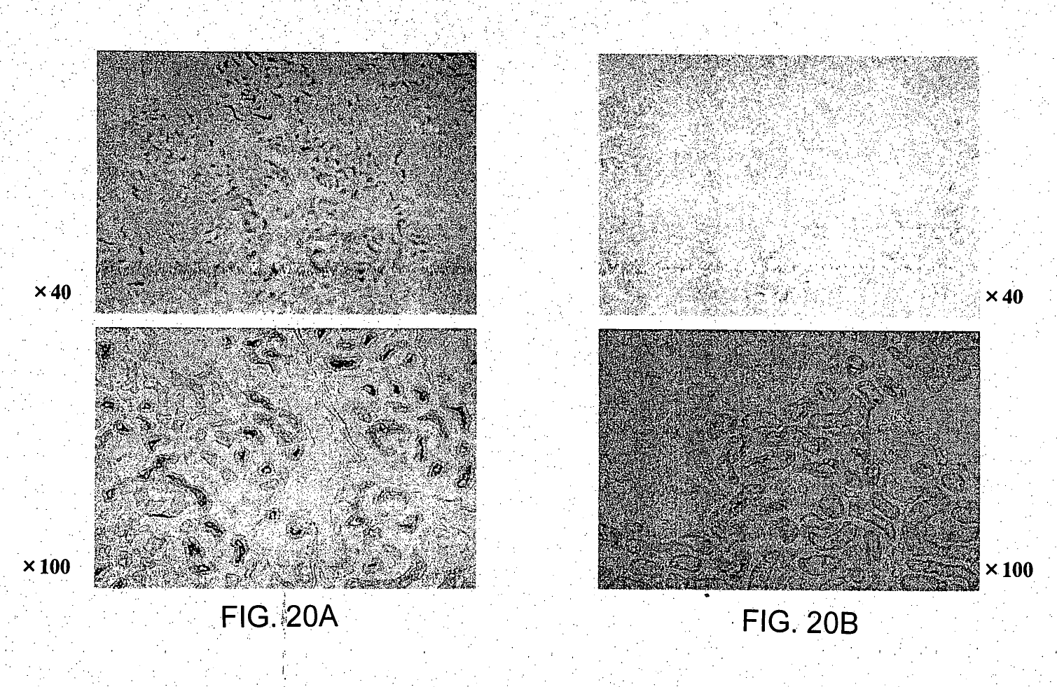

Immunohistochemical staining analysis was performed by a method similar to that of Example 18, except that a specific antibody against type IIc NaPi cotransporter was used as the antibody. Upon immunohistochemical staining using a specific antibody against type IIc NaPi cotransporter, expression of type IIc NaPi cotransporter significantly increased due to intake of low phosphorus diet. Expression in the brush border membrane significantly decreased in the FGF23Rl79Q-administered group, compared to that of the MOCK-administered group. The decrease was significant especially in the outer cortex, and some expression was observed in the deep cortex (FIG. 20).

1. An isolated protein selected from any one of the following (a) to (c):
(a) a protein comprising the amino acid sequence of SEQ ID NO:2;
(b) a protein comprising the amino acid sequence of SEQ ID NO:4; and
(c) a protein comprising the amino acid sequence of SEQ ID NO:2 or 4, wherein one or more amino acids have been deleted, substituted, or added, wherein the protein comprises Na/Pi cotransporter activity.

2. An isolated DNA encoding the protein of claim 1.

3. The DNA of claim 2 selected from any one of the following (a) to (c):
(a) a DNA comprising the nucleotide sequence of SEQ ID NO: 1;
(b) a DNA comprising the nucleotide sequence of SEQ ID NO:3; and
(c) a DNA that hybridizes under stringent conditions with a DNA comprising a nucleotide sequence complementary to the nucleotide sequence of SEQ ID NO: 1 or 3.

4. An isolated oligonucleotide comprising a nucleotide sequence comprising at least 15 continuous nucleotides of the nucleotide sequence of SEQ ID NO: 1 or 3.

5. A recombinant vector comprising the DNA of claim 2.

6. A transformant obtainable by transforming a host with the vector of claim 5.

7. A method of producing an Na/Pi cotransporter, wherein the method comprises culturing the transformant of claim 6, and collecting a protein comprising Na/Pi cotransport activity from the culture.

8. An antibody that reacts with the protein of claim 1.

9. A method of screening for substances comprising reactivity towards an Na/Pi cotransporter, wherein the method comprises the steps of (1) and (2):
(1) mixing a test substance with the protein of claim 1; and
(2) detecting binding between the protein and the test substance.

10. A method of screening for substances that regulate Na/Pi cotransporter expression, wherein the method comprises the steps of (1) and (2):
(1) adding a test substance to cells expressing the protein of claim 1, and culturing the cells; and
(2) measuring the protein of claim 1 expressed in the cells, or an mRNA encoding the protein.

11. A pharmaceutical composition for treating hypophosphatemia, wherein the composition comprises the DNA of claim 2.

12. A method of treating hypophosphatemia, wherein the method comprises the step of administering the DNA of claim 2 to a mammal.

13. A type II Na/Pi cotransporter expression regulatory factor selected from the proteins of any one of the following (a) to (d):
(a) an isolated protein comprising the amino acid sequence of SEQ ID NO:6;
(b) an isolated protein comprising the amino acid sequence of SEQ ID NO:6, wherein arginine at position 176 is replaced with glutamine;
(c) an isolated protein comprising the amino acid sequence of SEQ ID NO:6, wherein arginine at position 179 is replaced with glutamine; and (d) an isolated protein comprising an amino acid sequence of any one of the above-mentioned (a) to (c), wherein one or more amino acids have been deleted, substituted, added, or inserted.

14. A type II Na/Pi cotransporter expression modulator comprising as an active ingredient a DNA encoding a protein that can regulate expression of a type II Na/Pi cotransporter, wherein the DNA is selected from the following (a) and (b):
(a) an isolated DNA comprising the nucleotide sequence of SEQ ID NO:5; and
(b) an isolated DNA hybridizing under stringent conditions with a DNA comprising a nucleotide sequence complementary to the nucleotide sequence of SEQ ID NO:5.

15. The type II Na/Pi cotransporter expression modulator of claim 14, wherein the DNA is carried in a vector.

16. A pharmaceutical agent for the treatment of hyperphosphatemia, wherein the agent comprises, the type II Na/Pi cotransporter expression regulatory factor of claim 13 as an active ingredient.

17. A pharmaceutical agent for the treatment of hyperphosphatemia, wherein the agent comprises, the type II Na/Pi cotransporter expression modulator of claim 14 as an active ingredient.

18. A method of treating hyperphosphatemia, wherein the method comprises the step of administering the pharmaceutical agent of claim 16 to a patient.

19. A method of screening for substances that interact with a type II Na/Pi cotransporter expression regulatory factor comprising the following (A) and (B):
(A) reacting a test substance with the type II Na/Pi cotransporter expression regulatory factor of claim 13; and
(B) analyzing the presence or absence of interaction between the test substance and the type II Na/Pi cotransporter expression regulatory factor.

20. A recombinant vector comprising the DNA of claim 3.

21. A method of producing an Na/Pi cotransporter, wherein the method comprises culturing the transformant of claim 20, and collecting a protein comprising Na/Pi cotransport activity from the culture.

22. A pharmaceutical agent for the treatment of hyperphosphatemia, wherein the agent comprises the type II Na/Pi cotransporter expression modulator of claim 15 as an active ingredient.

23. A method of treating hyperphosphatemia, wherein the method comprises the step of administering the pharmaceutical agent of claim 17 to a patient.

24. A method of treating hyperphosphatemia, wherein the method comprises the step of administering the pharmaceutical agent of claim 22 to a patient.


SEQUENCE LISTING (21 sequences)

SEQ ID NO: 1 - DNA, 2026 bp, Homo sapiens
gtcgacccacgcgtccgcaccacccacacagatctagacctgggcctgggtctgtccctgcccgaaatccatgccgagttcccttcccggcagccaggtcccccaccccactctggacgcggttgacctagtggaaaagactctgaggaatgaagggacctccagttctgctccagtcttggaggaaggggacacagacccctggaccctccctcagctgaaggacacaagccagccctggaaagagctccgcgtggccggcaggctgcgccgcgtggccggcagcgtcctcaaggcctgcgggctcctcggcagcctgtacttcttcatctgctctctggacgtcctcagctccgccttccagctgctgggcagcaaagtggccggagacatcttcaaggacaacgtggtgctgtccaaccctgtggctggactggtcattggcgtgctggtcacagccctggtgcagagttccagcacgtcctcctccatcgtggtcagcatggtggctgctaagctgctgactgtccgggtgtctgtgcccatcatcatgggtgtcaacgtaggcacatccatcaccagcaccctggtctcaatggcgcagtcaggggaccgggatgaatttcagagggctttcagcggctcggcggtgcacgggatcttcaactggctcacagtgctggtcctgctgccactggagagcgccacggccctgctggagaggctaagtgagctagccctgggtgccgccagcctgacacccagggcgcaggcgcccgacatcctcaaggtgctgacgaagccgctcacacacctcatcgtgcagttggactccgacatgatcatgagcagtgccacaggcaacgccactaacagcagtctcattaagcactggtgcggcaccacggggcagccgacccaggagaacagcagctgtggcgccttcggcccgtgcacagagaagaacagcacagccccggcggacaggctgccctgccgccacctgtttgcgggcacggagctcacggacctggccgtgggctgcatcctgctggccggctccctgctggtgctctgcggctgcctggtcctcatagtcaagctgctcaactctgtgctgcgcggccgcgtggcccaggtcgtgaggacagtcatcaatgcggacttccccttcccgctgggctggctcggcggctacctggccgtcctcgcgggcgccggcctgaccttcgcactgcagagcagcagcgtcttcacggcggccgtcgtgcccctcatgggggtcggggtgatcagtctggaccgggcgtaccccctcttactgggctccaacatcggcaccactaccacagccctgctggctgccctggccagccccgcagacaggatgctcagcgccctgcaggtcgccctcatccacttcttcttcaacctggccggcatcctgctgtggtacctggtgcctgcactgcggctgcccatcccgctggccaggcacttcggggtggtgaccgcccgttaccgctgggtggctggggtctacctgctgctcggattcctgctgctgcccctggcggccttcgggctctccctggcagggggcatggtgctggccgctgtcgggggtcccctggtggggctggtgctcctcgtcatcctggttactgtcctgcagcggcgccggccggcctggctgcctgtccgcctgcgctcctgggcctggctccccgtctggctccattctctggagccctgggaccgcctggtgacccgctgctgcccctgcaacgtctgcagccccccgaaggccaccaccaaagaggcctactgctacgagaaccctgagatcttggcctcccagcagttgtgacgggcagttgctgagcagaccgccccaccctccccggctgggagggctctggagggccctggagggggggtccccgcggcagctgacctccggtcacctgcttccccttctgtgcaaataaaccaggctgttatctggaaaaaaaaaaaaaaaaaa

SEQ ID NO: 2 - protein, 599 aa, Homo sapiens
MPSSLPGSQVPHPTLDAVDLVEKTLRNEGTSSSAPVLEEGDTDPWTLPQLKDTSQPWKELRVAGRLRRVAGSVLKACGLLGSLYFFICSLDVLSSAFQLLGSKVAGDIFKDNVVLSNPVAGLVIGVLVTALVQSSSTSSSIVVSMVAAKLLTVRVSVPIIMGVNVGTSITSTLVSMAQSGDRDEFQRAFSGSAVHGIFNWLTVLVLLPLESATALLERLSELALGAASLTPRAQAPDILKVLTKPLTHLIVQLDSDMIMSSATGNATNSSLIKHWCGTTGQPTQENSSCGAFGPCTEKNSTAPADRLPCRHLFAGTELTDLAVGCILLAGSLLVLCGCLVLIVKLLNSVLRGRVAQVVRTVINADFPFPLGWLGGYLAVLAGAGLTFALQSSSVFTAAVVPLMGVGVISLDRAYPLLLGSNIGTTTTALLAALASPADRMLSALQVALIHFFFNLAGILLWYLVPALRLPIPLARHFGVVTARYRWVAGVYLLLGFLLLPLAAFGLSLAGGMVLAAVGGPLVGLVLLVILVTVLQRRRPAWLPVRLRSWAWLPVWLHSLEPWDRLVTRCCPCNVCSPPKATTKEAYCYENPEILASQQL

SEQ ID NO: 3 - DNA, 2075 bp, Rattus norvegicus
gtcgacgctgggcctcctccccgctggtgccccctcaccacacatgcagatctaggattgggcctgggtcttccacttcttgaaatccatgccgaattcccttgctggcgaccaggtccccaaccctactctggatgcaattggcctggtggactggagtctgagaaatgcagggacctctggttccaccccaggcttagaggagggaggcacagacccctggaccttctctcagctgaagaatactgaccaactcaaagaggttggcacagccagcaagctgcaccaggtggtcagcggcttcctcaaggcatgcggactcctgggaagcctctactttttcatctgctccctggacatcctcagctctgcgttccagctactaggcagcaagatggccggagacatttttaaggacaatgtggtgctgtccaaccctgtggctggcttggtcattggtgtggtggtcaccgtccttgtacagagctccagcacatcttcctctatcgtggtcagcatggtggcctctaagtcgctgactgtccaagcgtctgtccctatcatcatgggcgtcaacgtgggcacttccatcaccagcactctggtctcgatggcacagtcaggggatcgggatgaatttcagagggccttcggcggctcggccgtgcacggcatcttcaactggctcacagtgttggtactgctgccactggagaatgccactgcggcactagagagactgagtgagctggccctgggtgctgctagcctgcagccaggggggcaggcccccgacatcctcaaggcactgactcgacctttcacacacctcatcatccagctggatagcagtgtggttaccagcagcattaccagcaacaccactaacagcagcctgattaagcactggtgtggcttcaggggggagacgccccagggaagcagtgaggaatgcgacctctcgggctcttgcacagagaggaacagctcagcatctccaggagaggacagactgctctgccatcacctgtttgcaggctcagagctcacagacctggctgtgggcttcatcctgctggcaggttcgctgctggtgctctgtgtctgtctggtcctcatcgttaaactgctcaactctgtgctgcggggtcgcattgcacaggctgtgaagaccgttatcaatgcagattttcccttcccctttggctggctcagcggctacttggccatcctcgttggagcaggcttaaccttcttgcttcagagcagtagtgtcttcacggcagccattgtgcctctcatgggggttggggtgatcaacctggaacgggcctaccccctcttcctgggctccaacattggcaccaccaccacagccttactggctgccctggccagccctgcagacacgttactcttcgcagttcaggttgctctcatccacttctttttcaacctggctggcatactgctgtggtacctggtgcctgtcttgagactgccaattccattggccaagcgctttggggacctgactgcacaataccgctgggtagccattgtctacttactactcaccttccttctgctacccctagcagcctttggactttccctggctgggggttcagtgctggctgcagtggggggtcctctggtggggctggtactccttatcatcctggttaatgtcctgcaacgacaccgaccatcttggttgcctcgccgtcttcaatcctgggcctggctgccactttggctccattctctggagccctgggaccgcctggtgactggttgttgccccttcaaggcctacagcaactcccatatgaccagcaaagtggctcactgctatgagaacccacaagtcatagcttctcagcagttgtgagaagtgggtgcccatcaaaacccagttccactagcttcggggcattggaggcaggagaaaccactggccaatcttccctggggctgagtggcagggctccacatggatgttttagtctctgcatcctctccttttcaaataaacgaggttactattaaaaaaaaaaaaaaaaaaagtcgac

SEQ ID NO: 4 - protein, 601 aa, Rattus norvegicus
MPNSLAGDQVPNPTLDAIGLVDWSLRNAGTSGSTPGLEEGGTDPWTFSQLKNTDQLKEVGTASKLHQVVSGFLKACGLLGSLYFFICSLDILSSAFQLLGSKMAGDIFKDNVVLSNPVAGLVIGVVVTVLVQSSSTSSSIVVSMVASKSLTVQASVPIIMGVNVGTSITSTLVSMAQSGDRDEFQRAFGGSAVHGIFNWLTVLVLLPLENATAALERLSELALGAASLQPGGQAPDILKALTRPFTHLIIQLDSSVVTSSITSNTTNSSLIKHWCGFRGETPQGSSEECDLSGSCTERNSSASPGEDRLLCHHLFAGSELTDLAVGFILLAGSLLVLCVCLVLIVKLLNSVLRGRIAQAVKTVINADFPFPFGWLSGYLAILVGAGLTFLLQSSSVFTAAIVPLMGVGVINLERAYPLFLGSNIGTTTTALLAALASPADTLLFAVQVALIHFFFNLAGILLWYLVPVLRLPIPLAKRFGDLTAQYRWVAIVYLLLTFLLLPLAAFGLSLAGGSVLAAVGGPLVGLVLLIILVNVLQRHRPSWLPRRLQSWAWLPLWLHSLEPWDRLVTGCCPFKAYSNSHMTSKVAHCYENPQVIASQQL

SEQ ID NO: 5 - DNA, 756 bp, Homo sapiens
atgttgggggcccgcctcaggctctgggtctgtgccttgtgcagcgtctgcagcatgagcgtcctcagagcctatcccaatgcctccccactgctcggctccagctggggtggcctgatccacctgtacacagccacagccaggaacagctaccacctgcagatccacaagaatggccatgtggatggcgcaccccatcagaccatctacagtgccctgatgatcagatcagaggatgctggctttgtggtgattacaggtgtgatgagcagaagatacctctgcatggatttcagaggcaacatttttggatcacactatttcgacccggagaactgcaggttccaacaccagacgctggaaaacgggtacgacgtctaccactctcctcagtatcacttcctggtcagtctgggccgggcgaagagagccttcctgccaggcatgaacccacccccgtactcccagttcctgtcccggaggaacgagatccccctaattcacttcaacacccccataccacggcggcacacccggagcgccgaggacgactcggagcgggaccccctgaacgtgctgaagccccgggcccggatgaccccggccccggcctcctgttcacaggagctcccgagcgccgaggacaacagcccgatggccagtgacccattaggggtggtcaggggcggtcgagtgaacacgcacgctgggggaacgggcccggaaggctgccgccccttcgccaagttcatctag

SEQ ID NO: 6 - protein, 251 aa, Homo sapiens
MLGARLRLWVCALCSVCSMSVLRAYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTRSAEDDSERDPLNVLKPRARMTPAPASCSQELPSAEDNSPMASDPLGVVRGGRVNTHAGGTGPEGCRPFAKFI

SEQ ID NO: 7 - DNA, 43 bp, Artificial
  Description of Artificial Sequence Artificially Synthesized Primer Sequence
ggaattctcgagccaccatgttgggggcccgcctcaggctctg

SEQ ID NO: 8 - DNA, 35 bp, Artificial
  Description of Artificial Sequence Artificially Synthesized Primer Sequence
ggaattctcgagctactagatgaacttggcgaagg

SEQ ID NO: 9 - DNA, 20 bp, Artificial
  Description of Artificial Sequence Artificially Synthesized Primer Sequence
cacggcagcacacccggagc

SEQ ID NO: 10 - DNA, 20 bp, Artificial
  Description of Artificial Sequence Artificially Synthesized Primer Sequence
cacggcggcacacccagagc

SEQ ID NO: 11 - DNA, 20 bp, Artificial
  Description of Artificial Sequence Artificially Synthesized Primer Sequence
cacggcggcacacctggagc

SEQ ID NO: 12 - DNA, 20 bp, Artificial
  Description of Artificial Sequence Artificially Synthesized Primer Sequence
cacggcagcacacccagagc

SEQ ID NO: 13 - DNA, 20 bp, Artificial
  Description of Artificial Sequence Artificially Synthesized Primer Sequence
cacggcagcacacctggagc

SEQ ID NO: 14 - DNA, 16 bp, Artificial
  Description of Artificial Sequence Artificially Synthesized Primer Sequence
gtccagggtagaggcc

SEQ ID NO: 15 - DNA, 16 bp, Artificial
  Description of Artificial Sequence Artificially Synthesized Primer Sequence
ggcctctaccctggac

SEQ ID NO: 16 - DNA, 16 bp, Artificial
  Description of Artificial Sequence Artificially Synthesized Primer Sequence
attggcctggtggact

SEQ ID NO: 17 - DNA, 15 bp, Artificial
  Description of Artificial Sequence Artificially Synthesized Primer Sequence
agtccaccaggccaa

SEQ ID NO: 18 - DNA, 1097 bp, Rattus norvegicus
agcacctcgacatccatcattgtcagcatggtctcctctggcttgttggaggtgagctccgccattccgataatcatgggctccaacatcgggacctctgtcaccaacaccattgtggccctgatgcaggcgggggacaggactgacttcaggcgggcttttgcaggggcaacagtgcatgactgctttaactggctgtctgttctggtcctgctgcccctggaggctgccacgggctacctgcaccacgtcaccgggcttgtggttgcctccttcaacatccgtggtggccgtgatgcccctgaccttctcaaggttatcacagagcccttcacaaagcttatcatccagctggacaagtctgtgattaccagcattgccgtgggggatgagtccctgaggaatcacagtctcattcggatttggtgtcaaccagagacaaaagaggcttccacttctatgtccagggtagaggccattggcagccttgcaaacaccaccatggagaaatgcaaccatatcttcgtggatactgggctgcccgacctggccgtggggctcattctgctggcgggctccctggtggtcctgtgcacttgtcttatcctcctcgtcaagatgctcaactctctgctgaagggccaagtggccaatgtcatccagaaggtcatcaacacagacttcccagcccccttcacttgggtcacaggctactttgccatggtggtgggcgccagcatgacgtttgttgtccagagcagttccgtgttcacctcagccatcactccgctcattggcctgggtgtgatcagtattgagcgggcctaccctctcaccctgggctccaacattggtaccaccactacagccatcctggcagcactggccagccccagggagaagctctccagctcctttcagattgccctctgccacttcttcttcaacatctcgggcatcctcctgtggtacccgctgccctgcacacgtctgcccatccgcatggccaaggcactgggcaaacgcactgccaagtaccgctggtttgccgtcctctacctcctcgtgtgcttcctgctcctgccctcactggtgtt

SEQ ID NO: 19 - DNA, 436 bp, Rattus norvegicus
gaaatccatgccgaattcccttgctggcgaccaggtccccaaccctactctggatgcaattggcctggtggactggagtctgagaaatgcagggacctctggttccaccccaggcttagaggagggaggcacagacccctggaccttctctcagctgaagaatactgaccaactcaaagaggttggcacagccagcaagctgcaccaggtggtcagcggcttcctcaaggcatgcggactcctgggaagcctctactttttcatctgctccctggacatcctcagctctgcgttccagctactaggcagcaagatggccggagacatttttaaggacaatgtggtgctgtccaaccctgtggctggcttggtcattggtgtggtggtcaccgtccttgtacagagctccagcacatcttcctctatcgtggtcagc

SEQ ID NO: 20 - protein, 639 aa, Homo sapiens
MLSYGERLGSPAVSPLPVRGGHVMRGTAFAYVPSPQVLHRIPGTSAYAFPSLGPVALAEHTCPCGEVLERHEPLPAKLALEEEQKPESRLVPKLRQAGAMLLKVPLMLTFLYLFVCSLDMLSSAFQLAGGKVAGDIFKDNAILSNPVAGLVVGILVTVLVQSSSTSTSIIVSMVSSGLLEVSSAIPIIMGSNIGTSVTNTIVALMQAGDRTDFRRAFAGATVHDCFNWLSVLVLLPLEAATGYLHHITRLVVASFNIHGGRDAPDLLKIITEPFTKLIIQLDESVITSIATGDESLRNHSLIQIWCHPDSLQAPTSMSRAEANSSQTLGNATMEKCNHIFVDTGLPDLAVGLILLAGSLVLLCTCLILLVKMLNSLLKGQVAKVIQKVINTDFPAPFTWVTGYFAMVVGASMTFVVQSSSVFTSAITPLIGLGVISIERAYPLTLGSNIGTTTTAILAALASPREKLSSAFQIALCHFFFNISGILLWYPVPCTRLPIRMAKALGKRTAKYRWFAVLYLLVCFLLLPSLVFGISMAGWQVMVGVGTPFGALLAFVVLINVLQSRSPGHLPKWLQTWDFLPRWMHSLKPLDHLITRATLCCARPEPRSPPLPPRVFLEELPPATPSPRLALPAHHNATRL

SEQ ID NO: 21 - protein, 690 aa, Homo sapiens
MAPWPELGDAQPNPDKYLEGAAGQQPTAPDKSKETNKTDNTEAPVTKIELLPSYSTATLIDEPTEVDDPWNLPTLQDSGIKWSERDTKGKILCFFQGIGRLILLLGFLYFFVCSLDILSSAFQLVGGKMAGQFFSNSSIMSNPLLGLVIGVLVTVLVQSSSTSTSIVVSMVSSSLLTVRAAIPIIMGANIGTSITNTIVALMQVGDRSEFRRAFAGATVHDFFNWLSVLVLLPVEVATHYLEIITQLIVESFHFKNGEDAPDLLKVITKPFTKLIVQLDKKVISQIAMNDEKAKNKSLVKIWCKTFTNKTQINVTVPSTANCTSPSLCWTDGIQNWTMKNVTYKENIAKCQHIFVNFHLPDLAVGTILLILSLLVLCGCLIMIVKILGSVLKGQVATVIKKTINTDFPFPFAWLTGYLAILVGAGMTFIVQSSSVFTSALTPLIGIGVITIERAYPLTLGSNIGTTTTAILAALASPGNALRSSLQIALCHFFFNISGILLWYPIPFTRLPIRMAKGLGNISAKYRWFAVFYLIIFFFLIPLTVFGLSLAGWRVLVGVGVPVVFIIILVLCLRLLQSRCPRVLPKKLQNWNFLPLWMRSLKPWDAVVSKFTGCFQMRCCYCCRVCCRACCLLCGCPKCCRCSKCCEDLEEAQEGQDVPVKAPETFDNITISREAQGEVPASDSKTECTAL